(19) United States
(12) Patent Application Publication
(10) Pub. No.: US 2003/0096013 A1
(43) Pub. Date: May 22, 2003

(54) Preparation of submicron sized particles with polymorph control

(76) Inventors: Jane Werling, Arlington Heights, IL (US); James E. Kipp, Wauconda, IL (US); Rajaram Sriram, Glenview, IL (US); Mark J. Doty, Grayslake, IL (US)

Correspondence Address: BELL, BOYD & LLOYD LLC, P.O. Box 1135, Chicago, IL, 60690-1135, US

(21) Appl. No.: 10/246,802

(22) Filed: Sep. 17, 2002

Related U.S. Application Data
(63) Continuation-in-part of application No. 10/035,821, filed on Oct. 19, 2001.
Application No. 10/035,821 is a continuation-in-part of application No. 09/953,979, filed on Sep. 17, 2001.
Application No. 09/953,979 is a continuation-in-part of application No. 09/874,637, filed on Jun. 5, 2001.
(60) Provisional application No. 60/258,160, filed on Dec. 22, 2000.

(51) Int. Cl. A61K 9/14; B29B 9/00

(52) U.S. Cl. 424/489; 264/5

(57) ABSTRACT

The present invention provides a method of preparing particles with polymorph and size control of a pharmaceutical compound, the method including the steps of: (1) providing a pharmaceutical compound in a first phase; (2) seeding the compound; (3) causing a phase change in the pharmaceutical compound to a second phase of a desired polymorphic form; and (4) wherein the mean particle size of the particles is less than 7 μm.

### CROSS REFERENCE TO RELATED APPLICATIONS

This application is a continuation in part of application Ser. No. 10/035,821 filed Oct. 19, 2001, which is a continuation in part of application Ser. No. 09/953,979 filed Sep. 17, 2001 which is a continuation in part of application Ser. No. 09/874,637 filed Jun. 5, 2001, which claims priority from provisional application Ser. No. 60/258,160 filed Dec. 22, 2000, each of which is incorporated herein by reference and made a part hereof.

### BACKGROUND OF THE INVENTION

There is an ever increasing number of pharmaceutical drugs being formulated that are poorly soluble or insoluble in aqueous solutions. Such drugs provide challenges to delivering them in an injectable form such as through parenteral administration. Drugs that are insoluble in water can have significant benefits when formulated as a stable suspension of particles of less than three microns diameter. Accurate control of particle size is essential for safe and efficacious use of these formulations. Particles must be less than seven microns in diameter to safely pass through capillaries without causing emboli (Allen et al., 1987; Davis and Taube, 1978; Schroeder et al., 1978; Yokel et al., 1981).

One approach to delivering an insoluble drug is disclosed in U.S. Pat. No. 2,745,785. This patent discloses a method for preparing crystals of penicillin G suitable for parenteral administration. The method includes the step of recrystallizing the penicillin G from a formamide solution by adding water to reduce the solubility of the penicillin G. The '785 Patent further provides that the penicillin G particles can be coated with wetting agents such as lecithin, or emulsifiers, surface-active and defoaming agents, or partial higher fatty acid esters of sorbitan or polyoxyalkyklene derivatives thereof, or aryl alkyl polyether alcohols or salts thereof. The '785 patent further discloses micronizing the penicillin G with an air blast under pressure to form crystals ranging from about 5 to 20 microns.

Another approach is disclosed in U.S. Pat. No. 5,118,528 which discloses a process for preparing nanoparticles. The process includes the steps of: (1) preparing a liquid phase of a substance in a solvent or a mixture of solvents to which may be added one or more surfactants; (2) preparing a second liquid phase of a non-solvent or a mixture of non-solvents, the non-solvent is miscible with the solvent or mixture of solvents for the substance; (3) adding together the solutions of (1) and (2) with stirring; and (4) removing of unwanted solvents to produce a colloidal suspension of nanoparticles. The '528 Patent discloses that it produces particles of the substance smaller than 500 nm without the supply of energy. In particular the '528 Patent states that it is undesirable to use high energy equipment such as sonicators and homogenizers.

U.S. Pat. No. 4,826,689 discloses a method for making uniformly sized particles from water-insoluble drugs or other organic compounds. First, a suitable solid organic compound is dissolved in an organic solvent, and the solution can be diluted with a non-solvent. Then, an aqueous precipitating liquid is infused, precipitating non-aggregated particles with substantially uniform mean diameters. The particles are then separated from the organic solvent. Depending on the organic compound and the desired particle size, the parameters of temperature, ratio of non-solvent to organic solvent, infusion rate, stir rate, and volume can be varied according to the invention. The '689 Patent discloses this process forms a drug in a metastable state which is thermodynamically unstable and which eventually converts to a more stable crystalline state. The '689 discloses trapping the drug in a metastable state in which the free energy lies between that of the starting drug solution and the stable crystalline form. The '689 Patent discloses utilizing crystallization inhibitors (e.g., polyvinylpyrrolidinone) and surface-active agents (e.g., poly(oxyethylene)-co-oxypropylene) to render the precipitate stable enough to be isolated by centrifugation, membrane filtration or reverse osmosis.

U.S. Pat. Nos. 5,091,188; 5,091,187 and 4,725,442 disclose: (a) either coating small drug particles with natural or synthetic phospholipids or (b) dissolving the drug in a suitable lipophilic carrier and forming an emulsion stabilized with natural or semisynthetic phospholipids. One of the disadvantages of these formulations is that certain drug particles in suspension tend to grow over time because of the dissolution and reprecipitation phenomenon known as the “Oswald ripening.”

Another approach to providing insoluble drugs for parenteral delivery is disclosed in U.S. Pat. No. 5,145,684. The '684 Patent discloses the wet milling of an insoluble drug in the presence of a surface modifier to provide a drug particle having an average effective particle size of less than 400 nm. The '684 Patent discloses the surface modifier is adsorbed on the surface of the drug particle in an amount sufficient to prevent agglomeration into larger particles.

In yet another attempt to provide insoluble drugs for parenteral delivery is disclosed in U.S. Pat. No. 5,922,355. The '355 Patent discloses providing submicron sized particles of insoluble drugs using a combination of surface modifiers and a phospholipid followed by particle size reduction using techniques such as sonication, homogenization, milling, microfluidization, precipitation or recrystallization.

U.S. Pat. No. 5,780,062 discloses a method of preparing small particles of insoluble drugs by: (1) dissolving the drug in a water-miscible first solvent, (2) preparing a second solution of a polymer and an amphiphile in an aqueous second solvent in which the drug is substantially insoluble whereby a polymer/ amphiphile complex is formed and (3) mixing the solutions from the first and second steps to precipitate an aggregate of the drug and polymer/amphiphile complex.

U.S. Pat. No. 5,858,410 discloses a pharmaceutical nanosuspension suitable for parenteral administration. The '410 patent discloses subjecting at least one solid therapeutically active compound dispersed in a solvent to high pressure homogenization in a piston-gap homogenizer to form particles having an average diameter, determined by photon correlation spectroscopy (PCS), of 40 run to 100 nm, the proportion of particles larger than 5 μm in the total population being less than 0.1% (number distribution determined with a Coulter counter), without prior conversion into a melt, wherein the active compound is solid at room temperature and is insoluble, only sparingly soluble or moderately soluble in water, aqueous media and/or organic solvents. The Examples in the '410 Patent disclose jet milling prior to homogenization.

U.S. Pat. No. 4,997,454 discloses a method for making uniformly sized particles from solid compounds. The method of the '454 Patent includes the steps of dissolving the solid compound in a suitable solvent followed by infusing precipitating liquid thereby precipitating non-aggregated particles with substantially uniform mean diameter. The particles are then separated from the solvent.

One approach is directed to the production of suspended particles coated with protein. U.S. Pat. No. 5,916,596, issued to Desai et al., discloses the application of high shear to a mixture of an organic phase having a pharmacologically active agent dispersed therein and an aqueous medium containing a biocompatible polymer. The mixture is sheared in a high pressure homogenizer at a pressure in the range of from about 3,000 to 30,000 psi. The '596 patent provides that the mixture must contain substantially no surfactants because the combined use of a surfactant with a protein results in the formation of large, needle-like crystalline particles that increase in size during storage. See columns 17-18, example 4. Example 2 discloses that crude emulsion may be sonicated to produce nanoparticles ranging from 350-420 nanometers.

U.S. Pat. No. 5,560,933, issued to Soon-Shiong et al., discloses the formation of a polymeric shell around the water-insoluble drug for in vivo delivery. The method discloses the application of sonication to a mixture comprising a polymer-containing aqueous medium and a dispersing agent having a substantially water-insoluble drug dispersed therein. In this reference, sonication is used to drive the formation of disulfide bonds in the polymer, causing it to crosslink so as to produce a polymeric shell around the drug. Sonication is conducted for a time sufficient for the disulfide bonds to form.

In U.S. Pat. No. 5,665,383, Grinstaff et al. discloses the application of ultrasound to a single-phase, i.e., an aqueous medium, to encapsulate an immunostimulating agent within a polymeric shell for in vivo delivery. The ultrasound promotes crosslinking of the encapsulating agent by disulfide bonds to form the shell.

Another approach to preparing a water-insoluble drug for in vivo delivery centers on reducing the size of the particles that deliver the drug. In one such series of patents, which include U.S. Pat. Nos. 6,228,399; 6,086,376; 5,922,355; and 5,660,858, Parikh et al. discloses that sonication may be used to prepare microparticles of the water-insoluble compound. Of these patents, U.S. Pat. No. 5,922,355 discloses an improvement to a method that uses sonication for making the smaller particles. The improvement comprises mixing an active pharmacological agent with a phospholipid and surfactants in a single-phase aqueous system and applying energy to the system to produce the smaller particles.

U.S. Pat. No. 5,091,188, issued to Haynes, also discloses reducing the size of particles of a pharmacologically active water-insoluble drug and employing a lipid coating on the particles to confer a solid form. The '188 patent is directed to a pharmaceutical composition of an aqueous suspension of solid particles of the drug having a diameter of about 0.05 to about 10 microns. The lipid coating affixed to the surface of the particles contributes to their solid form. The composition is produced by adding the drug to water and then reducing the particle size within the aqueous suspension. Example 6 of this reference discloses the use of a pharmacologically acceptable oil which is selected for its inability to dissolve the crystalline drug. See column 16, lines 8-12.

Still another approach for preparing microparticles of a pharmacological agent focuses on the use of phase inversion principles. U.S. Pat. Nos. 6,235,224 B1 and 6,143,211, both issued to Mathiowitz et al., disclose the use of phase inversion phenomena to precipitate microencapsulated microparticles. The method includes mixing a polymer and a drug with a solvent. This mixture is introduced into an effective amount of a miscible nonsolvent, thereby causing spontaneous formation of the microencapsulated product.

Microprecipitation by pH shifting is another technology used to prepare dispersions of a nanoparticulate pharmaceutical agent. See, e.g., U.S. Pat. Nos. 5,665,331 and 5,662,883. This technology involves dissolving a pharmaceutical agent in an aqueous base which is then neutralized to form a dispersion.

In yet another approach, such as that disclosed in U.S. Pat. No. 5,766,635, issued to Spenlenhauer et al., nanoparticles have been prepared by dissolving a poly(ethylene) oxide and/or poly(propylene) oxide in an organic solvent, mixing the organic solution so formed with an aqueous solution to cause nanoparticles to precipitate out of solution, and microfluidizing the precipitated solution without the use of surfactants.

### SUMMARY OF THE INVENTION

The present invention relates to methods for preparing submicron sized particles of a pharmaceutical compound. The methods include additional steps for controlling the polymorphic form of the pharmaceutical compound to ultimately produce submicron sized particles in the desired size range and a desired polymorphic form. The methods can be divided into two general categories. The first category involves using crystal seeding techniques during a process for preparing nanoparticles by adding energy to pharmaceutical compound to produce sub-micron sized particles of the compound. The second category involves using crystal seeding techniques during a precipitation process for preparing sub-micron sized particles of the compound.

The present invention provides a method of preparing particles with polymorph and size control of a pharmaceutical compound. The method includes the steps of: providing a pharmaceutical compound in a first phase; seeding the compound; causing a phase change in the pharmaceutical compound to a second phase of a desired polymorphic form; and wherein the mean particle size of the particles is less than 7 μm. The term “phase” in this context means the state of the compound and includes gas, supercooled liquid, liquid, semicrystalline, crystalline, and other phases that are know in the art and combinations of the phases. A phase change includes converting the compound from, for example, a supercooled liquid to crystalline, a crystalline material having a first polymorphic to a crystalline material having a second polymorphic form different from the first polymorphic form. The term “phase” shall also be used to designate components of an emulsion such as aqueous phase and organic phase.

The present invention further provides a method for preparing submicron sized particles of a pharmaceutical compound. The method includes the steps of: (1) dissolving the pharmaceutical compound in a first solvent to form a first solution, (2) precipitating the pharmaceutical compound to form a presuspension, and (3) seeding the first solution or the presuspension.

The present invention further provides a method for preparing submicron sized particles of a pharmaceutical compound. The method includes the steps of: (1) dissolving the pharmaceutical compound in a first solvent to form a first solution; (2) mixing the first solution with a second solvent to precipitate the pharmaceutical compound as particles to form a presuspension, wherein the solubility of the pharmaceutical compound is greater in the first solvent than in the second solvent; (3) providing a seed compound to the first solution or the second solvent or the presuspension; (4) adding energy to the presuspension; and (5) wherein the particles have an average particle size of less than 500 nm.

The present invention further provides a method for preparing submicron sized particles of a pharmaceutical compound including the steps of: (1) adding a sufficient quantity of the pharmaceutical compound to a first solvent to create a supersaturated solution; (2) aging the supersaturated solution to form detectable crystals to create a seeding mixture; and (3) mixing the seeding mixture with a second solvent to precipitate the pharmaceutical compound to form a presuspension, wherein the pharmaceutical compound has a greater solubility in the first solvent than in the second solvent.

The present invention further provides a method for preparing a submicron sized suspension of a pharmaceutical compound having a desired polymorphic form. The method includes the steps of: (1) providing a suitable carrier for the pharmaceutical compound; (2) dispersing the pharmaceutical compound in the carrier to define a presuspension; (3) applying energy to the presuspension; and (4) seeding the presuspension to provide particles of the pharmaceutical compound having an average effective particle size of less than 500 nm and having the desired polymorphic form.

### BRIEF DESCRIPTION OF THE FIGURES

FIG. 1 is a photomicrograph of the amorphous particles in Example 1 prior to homogenization;

FIG. 2 is a photomicrograph of the particles in Example 1 after annealing by homogenization;

FIG. 3 is an X-Ray diffractogram of microprecipitated itraconazole in with polyethylene glycol-660 12-hydroxystearate before and after homogenization in Example 5;

FIG. 4 is a photomicrograph of carbamazepine crystals before homogenization in Example 6;

FIG. 5 is a photomicrograph of carbamazepine microparticulate after homogenization (Avestin C-50);

FIG. 6 is a schematic diagram of Microprecipitation Process for Prednisolone used in Examples 9-12;

FIG. 7 is a photomicrograph of prednisolone suspension before homogenization (Hoffman Modulation Contrast, 1250×magnification);

FIG. 8 is a photomicrograph of prednisolone suspension after homogenization (Hoffman Modulation Contrast, 1250×magnification);

FIG. 9 is a comparison of size distributions of nanosuspensions (Example 13 of this invention) and commercial fat emulsion;

FIG. 10a is an x-ray powder diffraction pattern for raw material itraconazole. FIG. 10b is an x-ray powder diffraction pattern for SMP-2-PRE. (Example 16);

FIG. 11a is a DSC trace for raw material itraconazole (Example 16). FIG. 11b is a DSC trace for SMP-2-PRE. (Example 16);

FIG. 12 is a DSC trace for SMP-2-PRE showing the melt of the less stable polymorph upon heating to 160° C., a recrystallization event upon cooling, and the subsequent melting of the more stable polymorph upon reheating to 180° C. (Example 16);

FIG. 13 is a comparison of the DSC traces of SMP-2-PRE samples after homogenization. Solid line=sample seeded with raw material itraconazole. Dashed line=unseeded sample. The solid line has been shifted by 1 W/g for clarity (Example 16);

FIG. 14 is a DSC trace showing the effect of seeding during precipitation. Dashed line=unseeded sample, solid line=sample seeded with raw material itraconazole. The unseeded trace (dashed line) has been shifted upward by 1.5 W/g for clarity. (Example 17);

FIG. 15 is a DSC trace showing the effect of seeding the drug concentrate through aging. Top x-ray diffraction pattern is for crystals prepared from fresh drug concentrate, and is consistent with the stable polymorph (see FIG. 10a). Bottom pattern is for crystals prepared from aged (seeded) drug concentrate, and is consistent with the metastable polymorph (see FIG. 10b). The top pattern has been shifted upward for clarity. (Example 18). (¶8);

FIG. 16a is the FTIR spectrum of the raw material of itraconazole, and FIG. 16b is the FTIR spectrum of SMP-2-PRE. (Example 16);

FIG. 17a shows a DSC trace for itraconazole having two endotherms indicating the presence of two polymorphs (Example 22); and

FIG. 17b shows a DSC trace for itraconazole after seeding and grinding with a mortar and pestle. (Example 22).

### DETAILED DESCRIPTION OF THE INVENTION

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

The present invention relates to methods for preparing submicron sized particles of pharmaceutical compounds. The methods include an additional step or steps for controlling the crystal structure of the pharmaceutical compound to ultimately produce submicron sized particles in the desired size range and a desired crystal structure. The additional step can be used in conjunction with any method for preparing nanoparticles including those methods where nanoparticles are prepared by applying mechanical energy to reduce the particle size and methods where nanoparticles are prepared by precipitation techniques.

The pharmaceutical compound is preferably insoluble or sparingly soluble in water. What is meant by the term “insoluble in water” is a solubility of the compound of less than 10 mg/mL in water, and preferably, less than 1 mg/mL in water. What is meant by the term pharmaceutical compound is pharmaceutically active compounds and pharmaceutical excipients. Pharmaceutically active compounds can be selected from various groups such as, but not limited to: antihyperlipidemics, antimicrobials, e.g., antibacterials such as sulfadiazine, antifungals such as itraconazole; non-steroidal anti-inflammatory drugs, e.g., indomethacin; antihypercholesteremic agents, e.g., probucol; and steroidal compounds, e.g., dexamethasone; immunosuppresants, e.g., cyclosporin A, tacrolimus, and mycophenolate mofetil.

Energy Addition Techniques for Forming Nanoparticles

In general, the method of preparing nanoparticles using energy addition techniques includes the step of adding the pharmaceutical compound, which sometimes shall be referred to as a drug, in bulk form to a suitable vehicle such as water or aqueous based solution containing one or more of the excipients set forth below or other liquid in which the pharmaceutical compound is not appreciably soluble to form a presuspension of the drug and adding energy to the presuspension to form a post suspension of drug particles having an average effective particle size of less than about 500 nm and more preferably less than about 400 nm and even more preferably less than about 300 nm or from about 50 nm to about 500 nm or any range or combination or subcombination of ranges therein. The particles size measurements can be made using techniques well known in the art such as by dynamic light scattering methods (e.g., photocorrelation spectroscopy, laser diffraction, low-angle laser light scattering (LALLS), medium-angle laser light scattering (MALLS), light obscuration methods (Coulter method, for example), rheology, or microscopy (light or electron) within the ranges set forth above).

The pharmaceutical compound will have a preferred polymorphic form and after the crystal seeding step a portion of the drug particles will be in the desired polymorphic form. The desired polymorphic form may be the same as that of the drug in bulk form or different from the bulk form. In a preferred form of the invention, greater than 50% of the particles of the post suspension will be in the desired polymorphic form and more preferably, greater than 70%, even more preferably, greater than 90%.

The step of adding energy includes mechanical grinding techniques such as pearl milling, a ball milling, hammer milling, fluid energy milling or wet grinding techniques such as those disclosed in U.S. Pat. No. 5,145,684, which is incorporated herein by reference and made a part hereof. The crystal seeding step which shall be discussed in detail below can be carried out at any point during grinding.

The adding energy techniques further includes subjecting the drug containing dispersion to high shear conditions including cavitation, shearing or impact forces utilizing a microfluidizer, piston gap homogenizer or counter current flow homogenizer such as disclosed in U.S. Pat. No. 5,091,188 which is incorporated herein by reference and made a part hereof. Suitable piston gap homogenizers are commercially available such as those sold under the product name EMULSIFLEX by Avestin, and French Pressure Cells sold by Spectronic Instruments. Suitable microfluidizers are available from Microfluidics Corp. The crystal seeding step described below can be conducted at any point during the process of subjecting the solution to high shear conditions and most preferably is conducted prior to the energy addition step.

The step of adding energy can also be accomplished using sonication techniques. The step of sonicating can be carried out with any suitable sonication device such as the Branson Model S-450A or Cole-Parmer 500/750 Watt Model. Such devices are well known in the industry. Typically the sonication device has a sonication horn or probe that is inserted into the drug containing solution to emit sonic energy into the solution. The sonicating device, in a preferred form of the invention, is operated at a frequency of from about 1 kHz to about 90 kHz and more preferably from about 20 kHz to about 40 kHz or any range or combination of ranges therein. The probe sizes can vary and preferably is in distinct sizes such as ½ inch or ¼ inch or the like. It may also be desirable to cool the solution during sonication to temperatures below room temperature. The crystal seeding step described below can be conducted at any point during the process of subjecting the solution to high shear conditions and most preferably is conducted before the energy addition step.

Precipitation Methods for Preparing Submicron Sized Particles

Any known precipitation method for preparing submicron sized particles or nanoparticles in the art can be used in conjunction with the seeding step in the present invention. The following is a description of examples of precipitation methods. The examples are for illustration purposes, and are not intended to limit the scope of the present invention.

Microprecipitation Methods

One example of a microprecipitation method is disclosed in U.S. Pat. No. 5,780,062, which is incorporated herein by reference and made a part hereof. The '062 patent discloses an organic compound precipitation process including: (i) dissolving the organic compound in a water-miscible first solvent; (ii) preparing a solution of polymer and an amphiphile in an aqueous second solvent and in which second solvent the organic compound is substantially insoluble whereby a polymer/amphiphile complex is formed; and (iii) mixing the solutions from steps (i) and (ii) so as to cause precipitation of an aggregate of the organic compound and the polymer/amphiphile complex. The polymorph control step discussed in detail below can be conducted during any of these steps. In a most preferred form of the invention, the polymorph control step is conducted at the time of mixing the solutions (iii).

Another example of a suitable precipitation process is disclosed in co-pending and commonly assigned U.S. Ser. Nos. 09/874,499; 09/874,799; 09/874,637; and 10/021,692, which are incorporated herein by reference and made a part hereof. The process disclosed includes the steps of: (1) dissolving an organic compound in a water miscible first organic solvent to create a first solution; (2) mixing the first solution with a second solvent or water to precipitate the organic compound to create a presuspension; and (3) adding energy to the presuspension in the form of high-shear mixing or heat to provide a stable form of the organic compound having the desired size ranges. One or more optional surface modifiers can be added to the first organic solvent or the second aqueous solution. The polymorph control step discussed in detail below can be conducted during any of these steps.

Emulsion Precipitation Methods

One suitable emulsion precipitation technique is disclosed in the co-pending and commonly assigned U.S. Ser. No. 09/964,273, which is incorporated herein by reference and is made a part hereof. In this approach, the process includes the steps of: (1) providing a multiphase system having an organic phase and an aqueous phase, the organic phase having a pharmaceutically effective compound therein; and (2) sonicating the system to evaporate a portion of the organic phase to cause precipitation of the compound in the aqueous phase and having an average effective particle size of less than about 2μm. The step of providing a multiphase system includes the steps of: (1) mixing a water immiscible solvent with the pharmaceutically effective compound to define an organic solution, (2) preparing an aqueous based solution with one or more surface active compounds, and (3) mixing the organic solution with the aqueous solution to form the multiphase system. The step of mixing the organic phase and the aqueous phase can include the use of piston gap homogenizers, colloidal mills, high speed stirring equipment, extrusion equipment, manual agitation or shaking equipment, microfluidizer, or other equipment or techniques for providing high shear conditions. The crude emulsion will have oil droplets in the water of a size of approximately less than 1 μm in diameter. The crude emulsion is sonicated to define a microemulsion and eventually to define a submicron sized particle suspension.

The polymorph control step discussed in detail below can be conducted during any of these steps. The polymorph control step can be taken prior to, or after sonicating the system. In a most preferred form of the invention, the polymorph control step is conducted during the sonicating step.

Another approach to preparing submicron sized particles is disclosed in co-pending and commonly assigned U.S. Ser. No. 10/183,035, which is incorporated herein by reference and made a part hereof. The process includes the steps of: (1) providing a crude dispersion of a multiphase system having an organic phase and an aqueous phase, the organic phase having a pharmaceutical compound therein; (2) providing energy to the crude dispersion to form a fine dispersion; (3) freezing the fine dispersion; and (4) lyophilizing the fine dispersion to obtain submicron sized particles of the pharmaceutical compound. The step of providing a multiphase system includes the steps of: (1) mixing a water immiscible solvent with the pharmaceutically effective compound to define an organic solution; (2) preparing an aqueous based solution with one or more surface active compounds; and (3) mixing the organic solution with the aqueous solution to form the multiphase system. The step of mixing the organic phase and the aqueous phase includes the use of piston gap homogenizers, colloidal mills, high speed stirring equipment, extrusion equipment, manual agitation or shaking equipment, microfluidizer, or other equipment or techniques for providing high shear conditions.

The polymorph control step discussed in detail below can be conducted during any of these steps. In a most preferred form of the invention, the polymorph control step is conducted at the mixing step (3) of the step of providing a multiphase system.

Solvent Anti-Solvent Precipitation

Suitable solvent anti-solvent precipitation technique is disclosed in U.S. Pat. Nos. 5,118,528 and 5,100,591 which are incorporated herein by reference and made a part hereof. The process includes the steps of: (1) preparing a liquid phase of a biologically active substance in a solvent or a mixture of solvents to which may be added one or more surfactants; (2) preparing a second liquid phase of a non-solvent or a mixture of non-solvents, the non-solvent is miscible with the solvent or mixture of solvents for the substance; (3) adding together the solutions of (1) and (2) with stirring; and (4) removing of unwanted solvents to produce a colloidal suspension of nanoparticles. The '528 Patent discloses that it produces particles of the substance smaller than 500 nm without the supply of energy.

The polymorph control step discussed in detail below can be conducted during any of these steps. In a most preferred form of the invention, the polymorph control step is conducted at step (3) prior to adding together the solutions (1) and (2).

Phase Inversion Precipitation

One suitable phase inversion precipitation is disclosed in U.S. Pat. Nos. 6,235,224, 6,143,211 and U.S. patent application No. 2001/0042932 which are incorporated herein by reference and made a part hereof. Phase inversion is a term used to describe the physical phenomena by which a polymer dissolved in a continuous phase solvent system inverts into a solid macromolecular network in which the polymer is the continuous phase. One method to induce phase inversion is by the addition of a nonsolvent to the continuous phase. The polymer undergoes a transition from a single phase to an unstable two phase mixture: polymer rich and polymer poor fractions. Micellar droplets of nonsolvent in the polymer rich phase serve as nucleation sites and become coated with polymer. The '224 patent discloses that phase inversion of polymer solutions under certain conditions can bring about spontaneous formation of discrete microparticles, including nanoparticles. The '224 patent discloses dissolving or dispersing a polymer in a solvent. A pharmaceutical agent is also dissolved or dispersed in the solvent. For the crystal seeding step to be effective in this process it is desirable the agent is dissolved in the solvent. The polymer, the agent and the solvent together form a mixture having a continuous phase, wherein the solvent is the continuous phase. The mixture is then introduced into at least tenfold excess of a miscible nonsolvent to cause the spontaneous formation of the microencapsulated microparticles of the agent having an average particle size of between 10 nm and 10 μm. The particle size is influenced by the solvent:nonsolvent volume ratio, polymer concentration, the viscosity of the polymer-solvent solution, the molecular weight of the polymer, and the characteristics of the solvent-nonsolvent pair. The process eliminates the step of creating microdroplets, such as by forming an emulsion, of the solvent. The process also avoids the agitation and/or shear forces.

The polymorph control step discussed in detail below can be conducted during any of these steps. In a most preferred form of the invention, the polymorph control step is conducted prior to or during the adding of the nonsolvent to the continuous phase.

pH Shift Precipitation

pH shift precipitation techniques typically include a step of dissolving a drug in a solution having a pH where the drug is soluble, followed by the step of changing the pH to a point where the drug is no longer soluble. The pH can be acidic or basic, depending on the particular pharmaceutical compound. The solution is then neutralized to form a presuspension of submicron sized particles of the pharmaceutcially active compound. One suitable pH shifting precipitation process is disclosed in U.S. Pat. No. 5,665,331, which is incorporated herein by reference and made a part hereof. The process includes the step of dissolving of the pharmaceutical agent together with a crystal growth modifier (CGM) in an alkaline solution and then neutralizing the solution with an acid in the presence of suitable surface-modifying surface-active agent or agents to form a fine particle dispersion of the pharmaceutical agent. The precipitation step can be followed by steps of diafiltration clean-up of the dispersion and then adjusting the concentration of the dispersion to a desired level. This process of reportedly leads to microcrystalline particles of Z-average diameters smaller than 400 nm as measured by photon correlation spectroscopy.

The polymorph control step discussed in detail below can be conducted during any of these steps. In a preferred form of the invention, the polymorph control step is conducted prior to or during the neutralizing step.

Other examples of pH shifting precipitation methods are disclosed in U.S. Pat. Nos. 5,716,642; 5,662,883; 5,560,932; and 4,608,278, which are incorporated herein by reference and are made a part hereof.

Infusion Precipitation Method

Suitable infusion precipitation techniques are disclosed in the U.S. Pat. Nos. 4,997,454 and 4,826,689, which are incorporated herein by reference and made a part hereof First, a suitable solid compound is dissolved in a suitable organic solvent to form a solvent mixture. Then, a precipitating nonsolvent miscible with the organic solvent is infused into the solvent mixture at a temperature between about −10° C. and about 100° C. and at an infusion rate of from about 0.01 ml per minute to about 1000 ml per minute per volume of 50 ml to produce a suspension of precipitated non-aggregated solid particles of the compound with a substantially uniform mean diameter of less tan10 μm. Agitation (e.g., by stirring) of the solution being infused with the precipitating nonsolvent is preferred. The nonsolvent may contain a surfactant to stabilize the particles against aggregation. The particles are then separated from the solvent. Depending on the solid compound and the desired particle size, the parameters of temperature, ratio of nonsolvent to solvent, infusion rate, stir rate, and volume can be varied according to the invention. The particle size is proportional to the ratio of nonsolvent:solvent volumes and the temperature of infusion and is inversely proportional to the infusion rate and the stirring rate. The precipitating nonsolvent may be aqueous or non-aqueous, depending upon the relative solubility of the compound and the desired suspending vehicle.

The polymorph control step discussed in detail below can be conducted during any of these steps. In a preferred form of the invention, the polymorph control step is conducted prior to or during the infusion of the nonsolvent.

Temperature Shift Precipitation

Temperature shift precipitation technique, also known as the hot-melt technique, is disclosed in U.S. Pat. No. 5,188,837 to Domb, which is incorporated herein by reference and made a part hereof. In an embodiment of the invention, lipospheres are prepared by the steps of: (1) melting or dissolving a substance such as a drug to be delivered in a molten vehicle to form a liquid of the substance to be delivered; (2) adding a phospholipid along with an aqueous medium to the melted substance or vehicle at a temperature higher than the melting temperature of the substance or vehicle; (3) mixing the suspension at a temperature above the melting temperature of the vehicle until a homogenous fine preparation is obtained; and then (4) rapidly cooling the preparation to room temperature or below.

The polymorph control step discussed in detail below can be conducted during any of these steps provided that the processing temperatures do not exceed the melting point of the drug. In a most preferred form of the invention, the polymorph control step is conducted before the step of cooling the warm drug dispersion.

Solvent Evaporation Precipitation

Solvent evaporation precipitation techniques are disclosed in U.S. Pat. No. 4,973,465 which is incorporated herein by reference and made a part hereof. The '465 Patent discloses methods for preparing microcrystals including the steps of: (1) providing a solution of a pharmaceutical composition and a phospholipid dissolved in a common organic solvent or combination of solvents, (2) evaporating the solvent or solvents and (3) suspending the film obtained by evaporation of the solvent or solvents in an aqueous solution by vigorous stirring. The solvent can be removed by adding energy to the solution to evaporate a sufficient quantity of the solvent to cause precipitation of the compound. The solvent can also be removed by other well known techniques such as applying a vacuum to the solution or blowing nitrogen over the solution. The polymorph control step discussed in detail below can be conducted during any of these steps. In a most preferred form of the invention, the polymorph control step is conducted prior to the evaporation step.

Reaction Precipitation

Reaction precipitation includes the steps of dissolving the pharmaceutical compound into a suitable solvent to form a solution. The compound should be added in an amount at or below the saturation point of the compound in the solvent. The compound is modified by reacting with a chemical agent or by modification in response to adding energy such as heat or UV light or the like to such that the modified compound has a lower solubility in the solvent and precipitates from the solution. The polymorph control step discussed in detail below can be conducted during any of these steps. In a most preferred form of the invention, the polymorph control step is conducted prior to or during the precipitation step.

Compressed Fluid Precipitation

A suitable technique for precipitating by compressed fluid is disclosed in WO 97/14407 to Johnston, which is incorporated herein by reference and made a part hereof. The method includes the steps of dissolving a water-insoluble drug in a solvent to form a solution. The solution is then sprayed into a compressed fluid, which can be a gas, liquid or supercritical fluid. The addition of the compressed fluid to a solution of a solute in a solvent causes the solute to attain or approach supersaturated state and to precipitate out as fine particles. In this case, the compressed fluid acts as an anti-solvent which lowers the cohesive energy density of the solvent in which the drug is dissolved.

Alternatively, the drug can be dissolved in the compressed fluid which is then sprayed into an aqueous phase. The rapid expansion of the compressed fluid reduces the solvent power of the fluid, which in turn causes the solute to precipitate out as fine particles in the aqueous phase. In this case, the compressed fluid acts as a solvent.

In order to stabilize the particles against aggregation, a surface modifier, such as a surfactant, is included in this technique. Particles prepared by this technique are generally 500 nm or smaller.

The polymorph control step discussed in detail below can be conducted during any of these steps. In a most preferred form of the invention, the polymorph control step is conducted prior to or during the particle formation step.

Polymorph Control

The present invention is directed to the preparation of submicron sized particles by one of the precipitation methods, such as the ones described above, with the particles having the desired crystal structure.

What is meant by the term “crystal structure” is the arrangement and/or conformation of the molecules within the crystal lattice. Compounds that can be crystallized into different crystal structures are said to be polymorphic. Numerous drug compounds and excipients used to deliver the drugs, set forth in detail below, are known to be polymorphic. Identification of polymorphs is an important step in drug formulation since different polymorphs of the same drug can show differences in solubility, therapeutic activity, bioavailability, and suspension stability. Similarly, different polymorphs of the same excipient can show differences in solubility, compatibility with the drug to be delivered, chemical stability and suspension stability. Accordingly, it is important to control the polymorphic form of the compound for ensuring product purity and batch-to-batch reproducibility.

The polymorphic form of the compound in the process discussed above can be controlled by the additional step of seeding. Seeding includes using a seed compound or adding energy to form a seed compound. In a preferred form of the invention, the seed compound is the pharmaceutically-active compound in the desired polymorphic form. Alternatively, the seed compound can also be an inert impurity or an organic compound with a structure similar to that of the desired polymorph.

The seed compound can be precipitated from a drug containing solution of any of the above-described processes. This method includes the steps of adding the pharmaceutically-active compound in sufficient quantity to exceed the solubility of the pharmaceutically-active compound in the first solution to create a supersaturated solution. The supersaturated solution is treated to precipitate the pharmaceutically-active compound in the desired polymorphic form. Treating the supersaturated solution includes aging the solution for a time period until the formation of a crystal or crystals is observed to create a seeding mixture. Treating the solution also includes subjecting the solution to temperature shifting or pH shifting. It is also possible to add energy to the supersaturated solution to cause the pharmaceutically-active compound to precipitate out of the solution in the desired polymorph. The energy can be added in a variety of ways including the energy addition steps described above. Further energy can be added by heating or exposing the presuspension to electromagnetic energy, particle beam or electron beam sources. The electromagnetic energy includes using a laser beam, dynamic electromagnetic energy, or other radiation sources It is further contemplated utilizing ultrasound, static electric field and a static magnetic field as the energy addition source.

In a preferred form of the invention, the method for producing seed crystals from an aged supersaturated solution includes the steps of: (i) adding a quantity of the pharmaceutically-active compound to a drug solution to create a supersaturated solution, (ii) aging the supersaturated solution to form detectable crystals to create a seeding mixture; and (iii) precipitating the seeding mixture to create a presuspension. The presuspension can then be further processed as described in detail above to provide an aqueous suspension of the pharmaceutically-active compound in the desired polymorph and in the desired size range.

Seeding can also be accomplished by adding energy to the first solution or the presuspension to form seed compound provided that the exposed liquid or liquids contain the pharmaceutical compound or a seed material. The energy can be added in the same fashion as described above for the supersaturated solution.

Accordingly, the present invention provides a composition of matter of a pharmaceutical compound in a desired polymorphic form essentially free of the unspecified polymorph or polymorphs. It is contemplated the methods of this invention can apply used to selectively produce a desired polymorph for numerous pharmaceutical compounds.

Optional Surface Active Compounds and Excipients

The drug solution or the presuspension or both the drug solution and the presuspension may be provided with one or more optional surface active compounds such as an anionic surfactant, a cationic surfactant, a nonionic surfactant or a biological surface active molecule added thereto. Suitable anionic surfactants include but are not limited to potassium laurate, sodium lauryl sulfate, sodium dodecylsulfate, alkyl polyoxyethylene sulfates, sodium alginate, dioctyl sodium sulfosuccinate, phosphatidyl choline, phosphatidyl glycerol, phosphatidyl inosine, phosphatidylserine, phosphatidic acid and their salts, glyceryl esters, sodium carboxymethylcellulose, cholic acid and other bile acids (e.g., cholic acid, deoxycholic acid, glycocholic acid, taurocholic acid, glycodeoxycholic acid) and salts thereof (e.g., sodium deoxycholate, etc.). Suitable cationic surfactants include but are not limited to quaternary ammonium compounds, such as benzalkonium chloride, cetyltrimethylammonium bromide, lauryldimethylbenzylammonium chloride, acyl camitine hydrochlorides, or alkyl pyridinium halides. As anionic surfactants, phospholipids may be used. Suitable phospholipids include, for example phosphatidylcholine, phosphatidylethanolamine, phosphatidylserine, phosphatidylinositol, phosphatidylglycerol, phosphatidic acid, lysophospholipids, egg or soybean phospholipid or a combination thereof. The phospholipid may be salted or desalted, hydrogenated or partially hydrogenated or natural semisynthetic or 1 5 synthetic.

Suitable nonionic surfactants include: polyoxyethylene fatty alcohol ethers (Macrogol and Brij), polyoxyethylene sorbitan fatty acid esters (Polysorbates), polyoxyethylene fatty acid esters (Myrj), sorbitan esters (Span), glycerol monostearate, polyethylene glycols, polypropylene glycols, cetyl alcohol, cetostearyl alcohol, stearyl alcohol, aryl alkyl polyether alcohols, polyoxyethylene-polyoxypropylene copolymers (poloxomers), polaxamines, methylcellulose, hydroxycellulose, hydroxy propylcellulose, hydroxy propylmethylcellulose, noncrystalline cellulose, polysaccharides including starch and starch derivatives such as hydroxyethylstarch (HES), polyvinyl alcohol, and polyvinylpyrrolidone. In a preferred form of the invention, the nonionic. surfactant is a polyoxyethylene and polyoxypropylene copolymer and preferably a block copolymer of propylene glycol and ethylene glycol. Such polymers are sold under the tradename POLOXAMER also sometimes referred to as PLURONIC®, and sold by several suppliers including Spectrum Chemical and Ruger. Among polyoxyethylene fatty acid esters is included those having short alkyl chains. One example of such a surfactant is SOLUTOL® HS 15, polyethylene-660-hydroxystearate, manufactured by BASF Aktiengesellschaft.

Surface active biological molecules include such molecules as albumin, casein, heparin, hirudin or other appropriate proteins.

It may also be desirable to add a pH adjusting agent to the second solution such as sodium hydroxide, hydrochloric acid, tris buffer or citrate, acetate, lactate, meglumine, or the like. The second solution should have a pH within the range of from about 3 to about 11.

Excipients known to be polymorphic include adeps solidus, amylodextrins, aspartam, butylhydroxyanisole, calcium oxalate, calcium phosphates, cellulose, chenodeoxycholic acid, cyclodextrins, fatty acids, fatty alcohols, glycerides, clyceromonostearate, glycine hydrochloride, hydrogenated canola oil, lactose, lipids, methol, magnesium stearate, paraffin, saccharose (sucrose), sorbitol, stearic acid, and suppository bases.

Suitable pharmaceutically active compounds which are known to be polymorphic include, but are not limited to, acebutolol hydrochloride, aceclidine hydrochloride, acedapsone, acemetacin, acetamide, acetaminophan, acetazolamide, acetohexamide, 21-acetoxypregnelonone, β-acetyldigoxin, DL-O-acetylpantolacton, acetylsalicylic acid, acetylsulisoxazole, adenosine derivative, adiphenine hydrochloride, ajmaline, allantoin, allobarbital, allopregnane-3β,20 -diol, 5-allyl-barbituric acid derivatives, alprenolol hydrochloride, amcinonid, amiloride hydrochloride, amino-acids, ρ-aminobenzoic acid, amikacin disulphate, aminopenicillanic acid derivatives, amiperone, amiflamine, amisometradine, amistriptyline hydrochloride, amobarbital, amoxilline, amphetamine sulphate, ampicillin, amylocaine hydrochloride, amrinone, androstane-diol derivatives, androstane-dione derivatives, androsterone, anilamate, anthranilic acid, anthraquinone carboxylic acid, aprindin hyrocholoride, aprobarbital, apronalide, arecoline hydrochloride, asparaginase, auronofin, azaperone, azelastine hydrochloride, azintamide, aztreonam, bacampicillin, baclofen, bamethan sulphate, bamipine hydrochloride, barbital, barbiturates, barbiturates azo derivatives, benactyzine, bendroflumethiazide, benoxaprfen benperiodol, bentiromide, benamide, benzilic acid esters, benzocaine, benzoylbenzoxazolidininone, benzopyran derivatives, benzoxalethiol, berberine hydrochloride, betamethasone acetate, bilamide, biotine, biperiden, bitoscanate, bolandiol dipropionate, bromisoval, bromovalerylurea, bromperidol, brompheniramine maleate, brotizolam, brucine, buclosamide, bumetanide, bupicomide,bupivacaine hydrochloride, bupranolol hydrochloride, busulphan, buspirone hydrochloride, butacaine hydrochloride, butallylonal, butinoline, buthalital sodium, butobarbital, butyxycaine hydrochloride, butropipazone, caffeine, calcium gluceptate, calcium lactate, calcium panthotenate, camphoric acid derivatives, captopril, caramiphen hydrochloride, carazolol, carbamazepine, carbocromen hydrochloride, carbromal, cofactor, cefaloridin, cefalotin sodium, cefamandole, cefazolin, cefixime, celirolot hydrochloride, chenodeozycholic acid, chloralhydrate, chloramphenicol derivatives, chloramphenicol palmitate, chlorbenzoamine dihydrochloride, chlordiazepoxide hydrochloride, chlorethyl aminouracil, chlormidazole hydrochloride, chloroacctamide, chlorphenoxamin hydrochloride, chlorpropamide, chlorpromazine hydrochloride, chloroquine diphosphate, chlorquinaidol, chlortestoterone, chlortetracycline hydrochloride, chlorthalidone, chlolesterol an esters, choline chloride, cibenzoline succinate, cimetidine, cinnamic acid, clenbuterol hydrochloride, clodantoin, clofenamine, clominorex, clomipramine hydrochloride, clonidin hydrochloride, clorindanol, clotrimazol, codeine, corticosterone, cortisone acetate, enanthate, coumaphol, cresols, crymoglycate disodium, cyclandelate, cyclobarbital, cyclobutyrol sodium, cyclopenthiazide cyclophosphamide, cyheptamide, cyproheptadine hydrochloride, danthron, daprone, dehydroandrosterone, dyhydroepiandrosterone, deoxycorticosterone propionate, descrpidine, dexamethasone acetate, dexamethasone palmitate, diacetylmorphine, diatrizoic acid, diazepam, dibromasalicyl, diclofenamide, diclofenac, dieclofenac aminosalicylate, dirovaltrate, diethylamine salicylate, diethylstilbestrol, difenoxin hydrochloride, dioctyl sodium sulphosuccinate, diphenadione, diphenidol, diphenylamine, diphenylhydantoin, diphenylmethane disulphonamide, diphenylprorenamine, diprophylline, dipyramidol, disopyramide, dobutamine hydrochloride, dormovit, doxylamine succinate, droloxifene, droperidol, embramine hydrochloride, emedastine difumarate, emetine hydrochloride, enalapril maleate, enoxaamine, ephedrine, epiandrosterone, eprozinol hydrochloride, ergometrine tartrate, ergotamine tartrate, erotamin tartrate, erythritol, erythromicin and estolate, estramucine, estradiol and salts, estradiol esters, estrone, etacrynic acid, etafedrine hydrochloride, ethallobarbital, ethambutol dihydrochloride, ethionamide, ethinyl estradiol, ethyl biscoumacetate, ethyl gallate, etidocaine hydrochloride, etiocholane derivatives, etofillne, etoposide, famotidine, felodipine, fenbufen, fendiline hydrochloride, fenoctimine sulphate, fenoterol hydrobromide, fenproporex, fenretinide, fluanisone, flucabril, flucloxacillin, fludrocortisone acetate, flufenamic acid, flumetramide, fluocortolone pivalate and salts, flucocortisol acetate, fluorohydrocortisone acetate, flupredniolone, fluogestone accetate, fluspirilene, fosinoprilsodium, fostedil, furaltadone, furosemide (frusemide), gepirone hydrochloride, glafenine, flibenclamide, flibomuride, flimidine (glycodiaine), dlucose, clutethimide, gramicidin, griscofulvin, fuaiphenesin, fuanoxyfen sulphate, halofenate, floperidol, hepabarbital, heptaminol hydrochloride, heptolamide, heroin (diacctylmorphine), hexachlorophene, heobarbital, histamine, histidine salts, homatripin hydrochloride, hydrochlorothiazide, hydrocortisone and salts, hydroflumethiazide, hydroxyphenylretinamide, hydroxyprocaine hydrochloride, hydroxypropyl theophylline, hyosscamine hydrochloride, hyoscine n-butylbromide, hyoscyamine sulphate, ibuprofen, ibuprofen lysinate, imidazopyridine derivatives, imidolin hydrochloride, imipramine hydrochloride, indalpin, indigo, indomethacin, inositol nicotinate, iopamidol, iopanoic acid, iprindole hydrochloride, isoajmalin, isomethadone hydrochloride, isoniazid, isoprenaline sulphate, isothiourea derivatives, ketoconazole, ketotifen hydrogen fumarate, dhellin, lovobunol, levodopa, levomepromazine hydrochloride, lidocaine hydrochloride, lisinopril, loperamide, lorazepam, lorcainide hydrochloride, losartan, mafenide hydrochloride, mebendazole, medrogestone, medetomidine hydrochloride, mefanamic acid, mefenorex hydrochloride, mefruside, menadione, menthol, mepacrine hydrochloride, mephemesine, mephentermine sulphate, mephesin carbamate, mepivacaine hydrochloride. meprobamate, mercaptopurine, mestranol, metahexamide, metalazon, metamphetamine, metaraminol bitartrate, melenolone, metformin hydrochloride, methadone, methallenestril, methamphetamine hydrochloride, methandriol and salts, methazole amide, methisazone, methoin, methoxsalen, methotrexate, methoxyphenylacetylosin, methylandrostanediol, methylestradiol, methyldopa, methylnitrovinylimidazole, methylphenylbarbituric acid, methylprednisolone and acetate, methylsulphanilysulphanilamide, methyltestoterone, metoclopramide and hydrochloride, metofenazate hydrochloride, metolazone, metronidazole benzoate, methyleinnamic acid, mexiletine hydrochloride, miconazole, midodrin hydrochloride, minoxidil hydrochloride, miokamycin, moclobemids, mefebutazone, moperone, mopidamol, morphine, mupirocin, nabilone, nafagrel hydrochloride. nafcillin, nafoxidin hydrochloride, naftifine hydrochloride, nalidixinic acid, nicametalte dihydrogencitrate, nicardipine hydrochloride, nicergoline, nicocodin, nicotinamide, nifedipine, nifenaolo hydrochloride, niflumic acid, nimodipin, nitrendipin, nitroflurmethan, nitrofurantoin, nordazepam, norethisterone, norfenfrine hydrochloride, norfloxacin, norleucine, norpseuodohedrin hydrochloride, nortriptyline hydrochloride, noscapin hydrochloride, novobiocin, noxiptylin, nystatin, ouaain, oxaceptrol. oxamniquine, oxazepam, oxeladin citrate, oxetacine, oxohexyltheobromide, oxprenolol hydrochloride, oxyclozanide, oxypendyl hydrochloride, oxyphenbutazone, oxytetracycline, pantolactone, paracetamol, paratoine, parsol 1789, paroxetine hydrochloride, penbutolol sulfate, n-pencillamine, penicillin G, pentamidine isethionate, pentazocine, pentobarbital, pentoxytylline, penoctone bromide, pethidine hydrochloride, phenacaine, phenacetine, phenadoxone hydrochloride, phenazine, phenazopyridine, phenelzine dihydrogensulphate, phenethylammonium bromide, phenformin hydrochloride, phenmetrazine and salts, phenobarbital, phenpromethamine hydrochloride, phensuximide, phentermine hydrochloride, phenylbutazone, phenylpropanolamine hydrochloride, pehnytoine, phthalysulphathiazole, pilocarpin nitrate, pimethixen, pimozide, pindolol, pipamperone, pipemidic acid, piperazine, piperylon, pipobroman, piribedil, piroxicam, pirprofen, polycaine hydrochloride, primidon, prodifen hydrochloride, probucol, progesterone, proline, promethazine, propallylonal, propantheline bromide, propanolol hydrochloride, propipocaine hydrochloride, propylhexedrine hydrochloride, propyphenazone, promethazine, propallylonal, propantheline bromide, propanolol hydrochloride, proscar, prothionamide, prothipendyl hydrochloride, proxibarbal, proxyphylline, pseudo-ephedrine hydrochloride, psilocin, psilocybin, pyramidon, pyrazinamide, pyrantel tartrate, pyridine derivatives, pyrimidine bases, pyrimidone derivatives, pyrisoxal hydrochloride, pyrithyldione, quercetin, quinine salts, raclopride tartrate, ramantidine, tentytolin hydrochloride, reserpin, resorantel, resorcinol, riboflavin, rifampicin, rotenone, solbutamol, salicylic acid, scopolamine hydrochloride, seebutobarbital, sodium ampicillin, sodium cromoglycate, spiperone, spironolactone, spiramycin, stadacacin, stanozolol, steroid hormones, streptomycin sulphate, stiripentol, succinylsulphathiazole, sulphabenzamide, sulphacetamide, sulphacarbamide, sulphacetamide, sulphachlorpyridazine, sulphadiazine, sulphadicramide, sulphadimidine, sulphaethidole, sulpha furazole, sulphaguanidine, sulphalene, sulphamerazine, sullphamethizole, sulphameter, sulphamethonium hydrochloride, sulphamethoxazole, sulphamethoxydiazine, sulphamethoxypyriazine, sulphamethylthiazole, sulphametrole, sulphamoxole, sulphamoyldiaminoazobenzene, sulphaanilamide, sulphanilamidomethoxypyrimidine, sulphanylxylamide, sulphapyrazole, sulphapyridine, sulphaproxiline, sulphathiazole, sulphathiourea, sulphatriazine, sulphazamet, sulphisoxazole, sulphonamides, sulphormetoxine, sulindae, suloetidil, sulpriride, tamoxifen citrate, terconazole, temazepam, terfenadine, terpin hydrate, testoterone and salts, tetracaine hydrochloride, tetracycline, tetrazolate derivatives, thebacon hydrochloride, theobroma oil, theophylline, thialbarbital, thiamine salts, thiamphenicol and salts, thiopental, thiosinamine, thiothyr, tiamizide, ticlopidine hydrochloride, tilidine hydrochloride, timolol maleate, tinidazole, tiocarilde, tobramycin, tobucarine hydrochloride, tolbutamic, tolropamine hydrochloride, tramazoline hydrochloride, tranilast, trazodone hydrochloride, triamcinolone diacetate, trimethoprim, trimetozine, triparanol, tromantadine hydrochloride, trospium hydrochloride, tyramine, uracil, uradipil, usninic acid, varbromal, verapamil hydrochloride, vesulong, vinbarbital, vitamin A acid, and voluntal.

### EXAMPLES

### Example 1

### Preparation of Itraconazole Suspension by use of Homogenization

To a 3-L flask add 1680 mL of Water for Injection, heat liquid to 60° C.-65° C., and then slowly add 44 grams of Pluronic F-68 (poloxamer 188), and 12 grams of sodium deoxycholate, stirring after each addition to dissolve the solids. After addition of solids is complete, stir for another 15 minutes at 60° C.-65° C. to ensure complete dissolution. Prepare a 50 mM tris (tromethamine) buffer by dissolving 6.06 grams of tris in 800 mL of Water for Injection. Titrate this solution to pH 8.0 with 0.1 M hydrochloric acid. Dilute the resulting solution to 1 liter with additional Water for Injection. Add 200 mL of the tris buffer to the poloxamer/deoxycholate solution. Stir thoroughly to mix solutions.

In a 150-mL beaker add 20 grams of itraconazole and 120 mL of N-methyl-2-pyrrolidinone. Heat mixture to 50° C.-60° C., and stir to dissolve solids. After total dissolution is visually apparent, stir another 15 minutes to ensure complete dissolution. Cool itraconazole-NMP solution to room temperature.

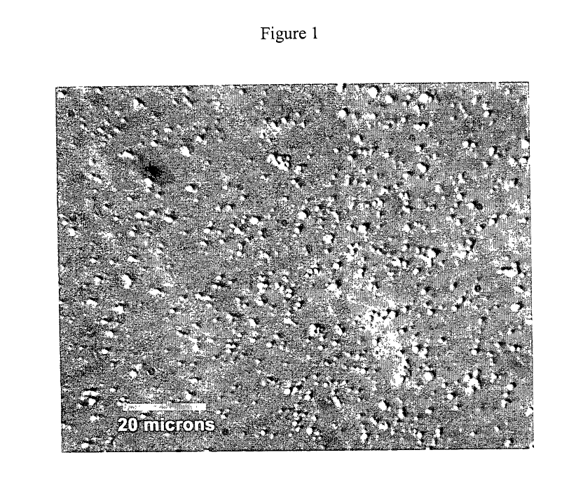

Charge a syringe pump (two 60-mL glass syringes) with the 120-mL of itraconazole solution prepared previously. Meanwhile pour all of the surfactant solution into a homogenizer hopper which has been cooled to 0° C.-5° C. (this may either by accomplished by use of a jacketed hopper through which refrigerant is circulated, or by surrounding the hopper with ice). Position a mechanical stirrer into the surfactant solution so that the blades are fully immersed. Using the syringe pump, slowly (1-3 mL/min) add all of the itraconazole solution to the stirred, cooled surfactant solution. A stirring rate of at least 700 rpm is recommended. An aliquot of the resulting suspension (Suspension A) is analyzed by light microscopy (Hoffman Modulation Contrast) and by laser diffraction (Horiba). Suspension A is observed by light microscopy to consist of roughly spherical amorphous particles (under 1 micron), either bound to each other in aggregates or freely moving by Brownian motion. See FIG. 1. Dynamic light scattering measurements typically afford a bimodal distribution pattern signifying the presence of aggregates (10-100 microns in size) and the presence of single amorphous particles ranging 200-700 nm in median particle diameter.

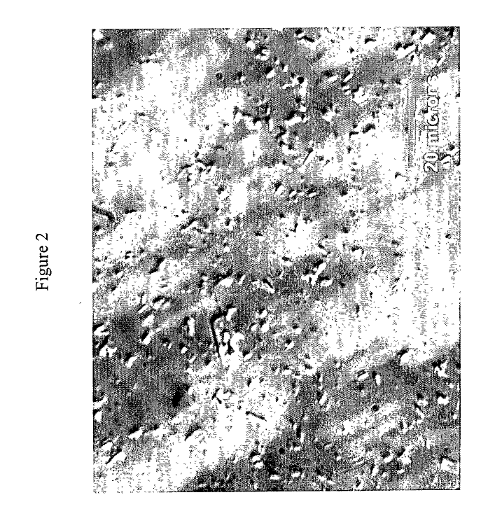

The suspension is immediately homogenized (at 10,000 to 30,000 psi) for 10-30 minutes. At the end of homogenization, the temperature of the suspension in the hopper does not exceed 75° C. The homogenized suspension is collected in 500-mL bottles, which are cooled immediately in the refrigerator (2° C.-8° C.). This suspension (Suspension B) is analyzed by light microscopy and is found to consist of small elongated plates with a length of 0.5 to 2 microns and a width in the 0.2-1 micron range. See FIG. 2. Dynamic light scattering measurements typically indicate a median diameter of 200-700 nm.

Stability of Suspension A (“Pre-suspension”) (Example 1)

During microscopic examination of the aliquot of Suspension A, crystallization of the amorphous solid was directly observed. Suspension A was stored at 2° C.-8° C. for 12 hours and examined by light microscopy. Gross visual inspection of the sample revealed severe flocculation, with some of the contents settling to the bottom of the container. Microscopic examination indicated the presence of large, elongated, plate-like crystals over 10 microns in length.

Stability of Suspension B

As opposed to the instability of Suspension A, Suspension B was stable at 2-8° C. for the duration of the preliminary stability study (1 month). Microscopy on the aged sample clearly demonstrated that no significant change in the morphology or size of the particles had occurred. This was confirmed by light scattering measurement.

### Example 2

### Preparation of Itraconazole Suspension by use of Ultrasonication

To a 500-mL stainless steel vessel add 252 mL of Water for Injection. Heat liquid to 60-65° C., and then slowly add 6.6 grams of Pluronic F-68 (poloxamer 188), and 0.9 grams of sodium deoxycholate, stirring after each addition to dissolve the solids. After addition of solids is complete, stir for another 15 minutes at 60-65° C. to ensure complete dissolution. Prepare a 50 mM tris (tromethamine) buffer by dissolving 6.06 grams of tris in 800 mL of Water for Injection. Titrate this solution to pH 8.0 with 0.1 M hydrochloric acid. Dilute the resulting solution to 1 liter with additional Water for Injection. Add 30 mL of the tris buffer to the poloxamer/deoxycholate solution. Stir thoroughly to mix solutions.

In a 30-mL container add 3 grams of itraconazole and 18 mL of N-methyl-2-pyrrolidinone. Heat mixture to 50° C.-60° C., and stir to dissolve solids. After total dissolution is visually apparent, stir another 15 minutes to ensure complete dissolution. Cool itraconazole-NMP solution to room temperature.

Charge a syringe pump with 18-mL of itraconazole solution prepared in a previous step. Position a mechanical stirrer into the surfactant solution so that the blades are fully immersed. Cool the container to 0° C.-5° C. by immersion in an ice bath. Using the syringe pump, slowly (1-3 mL/min) add all of the itraconazole solution to the stirred, cooled surfactant solution. A stirring rate of at least 700 rpm is recommended. Immerse an ultrasonicator horn in the resulting suspension so that the probe is approximately 1 cm above the bottom of the stainless steel vessel. Sonicate (10,000 to 25,000 Hz, at least 400W) for 15 to 20 minute in 5-minute intervals. After the first 5-minute sonication, remove the ice bath and proceed with further sonication. At the end of ultrasonication, the temperature of the suspension in the vessel does not exceed 75° C.

The suspension is collected in a 500-mL Type I glass bottle, which is cooled immediately in the refrigerator (2° C.-8° C.). Characteristics of particle morphology of the suspension before and after sonication were very similar to that seen Example 1 before and after homogenization, respectively.

### Example 3

### Preparation of Itraconazole Suspension by use of Homogenization

Prepare a 50 mM tris (tromethamine) buffer by dissolving 6.06 grams of tris in 800 mL of Water for Injection. Titrate this solution to pH 8.0 with 0.1 M hydrochloric acid. Dilute the resulting solution to 1 liter with additional Water for Injection. To a 3-L flask add 1680 mL of Water for Injection. Add 200 mL of the tris buffer to the 1680 mL of water. Stir thoroughly to mix solutions.

In a 150-mL beaker add 44 grams of Pluronic F-68 (poloxamer 188) and 12 grams of sodium deoxycholate to 120 mL of N-methyl-2-pyrrolidinone. Heat the mixture to 50-60° C., and stir to dissolve solids. After total dissolution is visually apparent, stir another 15 minutes to ensure complete dissolution. To this solution, add 20 grams of itraconazole, and stir until totally dissolved. Cool the itraconazole-surfactant-NMP solution to room temperature.

Charge a syringe pump (two 60-mL glass syringes) with the 120-mL of the concentrated itraconazole solution prepared previously. Meanwhile pour the diluted tris buffer solution prepared above into a homogenizer hopper which has been cooled to 0° C.-5° C. (this may either by accomplished by use of a jacketed hopper through which refrigerant is circulated, or by surrounding the hopper with ice). Position a mechanical stirrer into the buffer solution so that the blades are fully immersed. Using the syringe pump, slowly (1-3 mL/min) add all of the itraconazole-surfactant concentrate to the stirred, cooled buffer solution. A stirring rate of at least 700 rpm is recommended. The resulting cooled suspension is immediately homogenized (at 10,000 to 30,000 psi) for 10-30 minutes. At the end of homogenization, the temperature of the suspension in the hopper does not exceed 75° C.

The homogenized suspension is collected in 500-mL bottles, which are cooled immediately in the refrigerator (2° C.-8° C.). Characteristics of particle morphology of the suspension before and after homogenization were very similar to that seen in Example 1, except that in this process, the pre-homogenized material tended to form fewer and smaller aggregates which resulted in a much smaller overall particle size as measured by laser diffraction. After homogenization, dynamic light scattering results were typically identical to those presented in Example 1.

### Example 4

### Preparation of Itraconazole Suspension by use of Ultrasonication

To a 500-mL flask add 252 mL of Water for Injection. Prepare a 50 mM tris (tromethamine) buffer by dissolving 6.06 grams of tris in 800 mL of Water for Injection. Titrate this solution to pH 8.0 with 0.1 M hydrochloric acid. Dilute the resulting solution to 1 liter with additional Water for Injection. Add 30 mL of the tris buffer to the water. Stir thoroughly to mix solutions.

In a 30-mL beaker add 6.6 grams of Pluronic F-68 (poloxamer 188) and 0.9 grams of sodium deoxycholate to 18 mL of N-methyl-2-pyrrolidinone. Heat the mixture to 50° C.-60° C., and stir to dissolve solids. After total dissolution is visually apparent, stir another 15 minutes to ensure complete dissolution. To this solution, add 3.0 grams of itraconazole, and stir until totally dissolved. Cool the itraconazole-surfactant-NMP solution to room temperature.

Charge a syringe pump (one 30-mL glass syringe with the 18-mL of the concentrated itraconazole solution prepared previously. Position a mechanical stirrer into the buffer solution so that the blades are fully immersed. Cool the container to 0° C.-5° C. by immersion in an ice bath. Using the syringe pump, slowly (1-3 mL/min) add all of the itraconazole-surfactant concentrate to the stirred, cooled buffer solution. A stirring rate of at least 700 rpm is recommended. The resulting cooled suspension is immediately sonicated (10,000 to 25,000 Hz, at least 400 W) for 15-20 minutes, in 5-minute intervals. After the first 5-minute sonication, remove the ice bath and proceed with further sonication. At the end of ultrasonication, the temperature of the suspension in the hopper does not exceed 75° C.

The resultant suspension is collected in a 500-mL bottle, which is cooled immediately in the refrigerator (2° C.-8° C.). Characteristics of particle morphology of the suspension before and after sonication were very similar to that seen in Example 1, except that in this process, the pre-sonicated material tended to form fewer and smaller aggregates which resulted in a much smaller overall particle size as measured by laser diffraction. After ultrasonication, dynamic light scattering results were typically identical to those presented in Example 1.

### Example 5

### Preparation of Itraconazole Suspension (1%) with 0.75% Solutol® HR (PEG-660 12-Hydroxystearate).

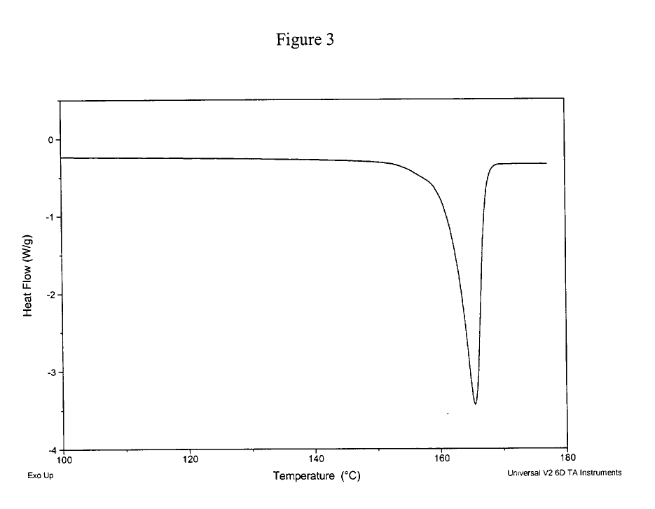

Solutol (2.25 g) and itraconazole (3.0 g) were weighed into a beaker and 36 mL of filtered N-methyl-2-pyrrolidinone (NMP) was added. This mixture was stirred under low heat (up to 40° C.) for approximately 15 minutes until the solution ingredients were dissolved. The solution was cooled to room temperature and was filtered through a 0.2-micron filter under vacuum. Two 60-mL syringes were filled with the filtered drug concentrate and were placed in a syringe pump. The pump was set to deliver approximately 1 mL/min of concentrate to a rapidly stirred (400 rpm) aqueous buffer solution. The buffer solution consisted of 22 g/L of glycerol in 5 mM tris buffer. Throughout concentrate addition, the buffer solution was kept in an ice bath at 2° C.-3° C. At the end of the precipitation, after complete addition of concentrate to the buffer solution, about 100 mL of the suspension was centrifuged for 1 hour, the supernatant was discarded. The precipitate was resuspended in a 20% NMP solution in water, and again centrifuged for 1 hour. The material was dried overnight in a vacuum oven at 25° C. The dried material was transferred to a vial and analyzed by X-ray diffractometry using chromium radiation (see FIG. 3).

Another 100 mL-aliquot of the microprecipitated suspension was sonicated for 30 minutes at 20,000 Hz, 80% full amplitute (full amplitude=600 W). The sonicated sample was homogenized in 3 equal aliquots each for 45 minutes (Avestin C5, 2° C.-5° C., 15,000-20,000 psi). The combined fractions were centrifuged for about 3 hours, the supernatant removed, and the precipitate resuspended in 20% NMP. The resuspended mixture was centrifuged again (15,000 rpm at 5° C.). The supernatant was decanted off and the precipitate was vacuum dried overnight at 25° C. The precipitate was submitted for analysis by X-ray diffractometry (see FIG. 3). As seen in FIG. 3, the X-ray diffraction patterns of processed samples, before and after homogenization, are essentially identical, yet show a significantly different pattern as compared with the starting raw material. The unhomogenized suspension is unstable and agglomerates upon storage at room temperature. The stabilization that occurs as a result of homogenization is believed to arise from rearrangement of surfactant on the surface of the particle. This rearrangement should result in a lower propensity for particle aggregation.

### Example 6

### Preparation of Carbamazepine Suspension by use of Homogenization

2.08 g of carbamazepine was dissolved into 10 mL of NMP. 1.0 mL of this concentrate was subsequently dripped at 0.1 mL/min into 20 mL of a stirred solution of 1.2% lecithin and 2.25% glycerin. The temperature of the lecithin system was held at 2-5° C. during the entire addition. The predispersion was next homogenized cold (5-15° C.) for 35 minutes at 15,000 psi. The pressure was increased to 23,000 psi and the homogenization was continued for another 20 minutes. The particles produced by the process had a mean diameter of 0.881 μm with 99% of the particles being less than 2.44 μm.

### Example 7

### Preparation of 1% Carbamazepine Suspension with 0.125% Solutol® by use of Homogenization

A drug concentrate of 20% carbamazepine and 5% glycodeoxycholic acid (Sigma Chemical Co.) in N-methyl-2-pyrrolidinone was prepared. The microprecipitation step involved adding the drug concentrate to the receiving solution (distilled water) at a rate of 0.1 mL/min. The receiving solution was stirred and maintained at approximately 5° C. during precipitation. After precipitation, the final ingredient concentrations were 1% carbamazepine and 0.125% Solutol®. The drug crystals were examined under a light microscope using positive phase contrast (400×). The precipitate consisted of fine needles approximately 2 microns in diameter and ranging from 50-150 microns in length.

Homogenization (Avestin C-50 piston-gap homogenizer) at approximately 20,000 psi for approximately 15 minutes results in small particles, less than 1 micron in size and largely unaggregated. Laser diffraction analysis (Horiba) of the homogenized material showed that the particles had a mean size of 0.4 micron with 99% of the particles less than 0.8 micron. Low energy sonication, suitable for breaking agglomerated particles, but not with sufficient energy to cause a cominution of individual particles, of the sample before Horiba analysis had no effect on the results (numbers were the same with and without sonication). This result was consistent with the absence of particle agglomeration.

Samples prepared by the above process were centrifuged and the supernatant solutions replaced with a replacement solution consisting of 0.125% Solutol®. After centrifugation and supernatant replacement, the suspension ingredient concentrations were 1% carbamazepine and 0.125% Solutol®. The samples were re-homogenized by piston-gap homogenizer and stored at 5° C. After 4 weeks storage, the suspension had a mean particle size of 0.751 with 99% less than 1.729. Numbers reported are from Horiba analysis on unsonicated samples.

### Example 8

### Preparation of 1% Carbamazepine Suspension with 0.06% Sodium Glycodeoxycholate and 0.06% Poloxamer 188 by use of Homogenization

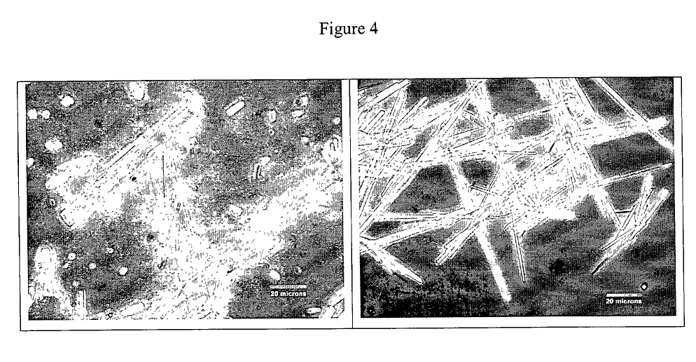

A drug concentrate comprising 20% carbamazepine and 5% glycodeoxycholate in N-methyl-2-pyrrolidinone was prepared. The microprecipitation step involved adding the drug concentrate to the receiving solution (distilled water) at a rate of 0.1 mL/min. Thus, this and the following examples demonstrate that adding a surfactant or other excipient to the aqueous precipitating solution in Methods A and B above is optional. The receiving solution was stirred and maintained at approximately 5° C. during precipitation. After precipitation, the final ingredient concentrations were 1% carbamazepine and 0.125% Solutol®. The drug crystals were examined under a light microscope using positive phase contrast (400×). The precipitate consisted of fine needles approximately 2 microns in diameter and ranging from 50-150 microns in length. Comparison of the precipitate with the raw material before precipitation reveals that the precipitation step in the presence of surface modifier (glycodeoxycholic acid) results in very slender crystals that are much thinner than the starting raw material (see FIG. 4).

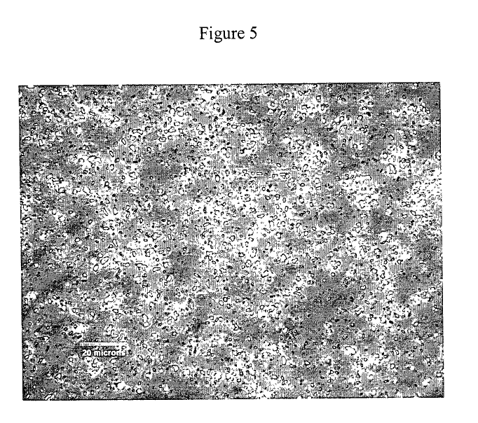

Homogenization (Avestin C-50 piston-gap homogenizer) at approximately 20,000 psi for approximately 15 minutes results in small particles, less than 1 micron in size and largely unaggregated. See FIG. 5. Laser diffraction analysis (Horiba) of the homogenized material showed that the particles had a mean size of 0.4 micron with 99% of the particles less than 0.8 micron. Sonication of the sample before Horiba analysis had no effect on the results (numbers were the same with and without sonication). This result was consistent with the absence of particle agglomeration.

Samples prepared by the above process were centrifuged and the supernatant solutions replaced with a replacement solution consisting of 0.06% glycodeoxycholic acid (Sigma Chemical Co.) and 0.06% Poloxamer 188. The samples were re-homogenized by piston-gap homogenizer and stored at 5° C. After 2 weeks storage, the suspension had a mean particle size of 0.531 micron with 99% less than 1.14 micron. Numbers reported are from Horiba analysis on unsonicated samples.

Mathematical Analysis (Example 8) of force required to break precipitated particles as compared to force required to break particles of the starting raw material (carbamazepine):

The width of the largest crystals seen in the carbamazepine raw material (FIG. 4, picture on left) are roughly 10-fold greater than the width of crystals in the microprecipitated material (FIG. 4, picture on right). On the assumption that the ratio of crystal thickness (1:10) is proportional to the ratio of crystal width (1:10), then the moment of force required to cleave the larger crystal in the raw material should be approximately 1,000-times greater than the force needed to break the microprecipitated material, since:

e_{L}=6PL/(Ewx²) Eq. 1

where,

e_{L}=longitudinal strain required to break the crystal (“yield value”)

P=load on beam

L=distance from load to fulcrum

E=elasticity modulus

w=width of crystal

x=thickness of crystal

Let us assume that L and E are the same for the raw material and the precipitated material. Additionally, let us assume that w/w₀=x/x₀=10. Then,

(e_{L})₀=6P₀L/(Ew₀x₀²), where the ‘0’ subscripts refer to raw material

e_{L}=6PL/(Ewx²), for the microprecipitate

Equating (e_{L})₀ and e_{L},

6PL/(Ewx²)=6P₀L/(Ew₀x₀²)

After simplification,

P=P₀(w/w₀) (x/x₀)²=P₀(0.1) (0.1)²=0.001 P₀

Thus, the yield force, P, required to break the microprecipitated solid is one-thousandth the required force necessary to break the starting crystalline solid. If, because of rapid precipitation, lattice defects or amorphic properties are introduced, then the modulus (E) should decrease, making the microprecipitate even easier to cleave.

### Example 9

### Preparation of 1.6% (w/v) Prednisolone Suspension with 0.05% Sodium Deoxycholate and 3% N-methyl-2-pyrrolidinone

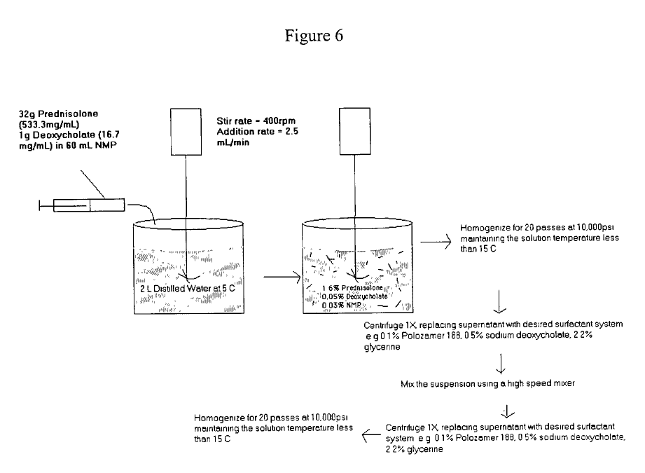

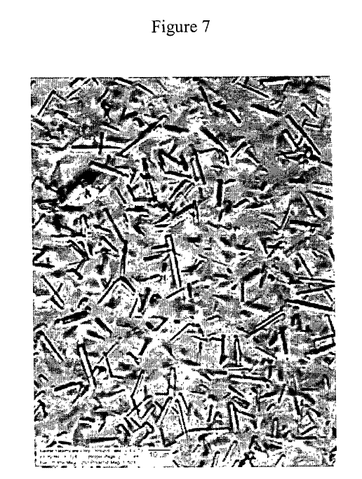

A schematic of the overall manufacturing process is presented in FIG. 6. A concentrated solution of prednisolone and sodium deoxycholate was prepared. Prednisolone (32 g) and sodium deoxycholate (1 g) were added to a sufficient volume of 1-methyl 2-pyrrolidinone (NMP) to produce a final volume of 60 mL. The resulting prednisolone concentration was approximately 533.3 mg/mL and the sodium deoxycholate concentration was approximately 16.67 mg/mL. 60 mL of NMP concentrate was added to 2 L of water cooled to 5° C. at an addition rate of 2.5 mL/min while stirring at approximately 400 rpm. The resulting suspension contained slender needle-shaped crystals less than 2 μm in width (FIG. 7). The concentration contained in the precipitated suspension was 1.6% (w/v) prednisolone, 0.05% sodium deoxycholate, and 3% NMP.

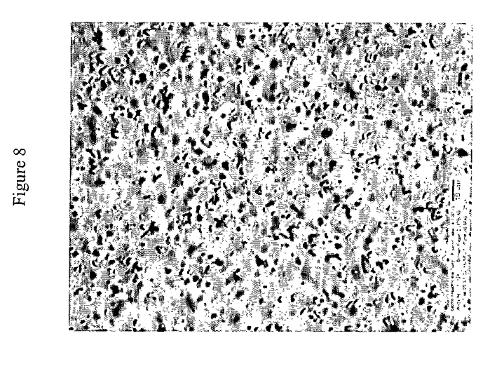

The precipitated suspension was pH adjusted to 7.5-8.5 using sodium hydroxide and hydrochloric acid then homogenized (Avestin C-50 piston-gap homogenizer) for 10 passes at 10,000 psi. The NMP was removed by performing 2 successive centrifugation steps replacing the supernatant each time with a fresh surfactant solution, which contained the desired concentrations of surfactants needed to stabilize the suspension (see Table 1). The suspension was homogenized for another 10 passes at 10,000 psi. The final suspension contained particles with a mean particle size of less than 1 μm, and 99% of particles less than 2 μm. FIG. 8 is a photomicrograph of the final prednisolone suspension after homogenization.

A variety of different surfactants at varying concentrations were used in the centrifugation/surfactant replacement step (see Table 1). Table 1 lists combinations of surfactants that were stable with respect to particle size (mean <1 μm, 99%<2 μm), pH (6-8), drug concentration (less than 2% loss) and re-suspendability (resuspended in 60 seconds or less).

Notably this process allows for adding the active compound to an aqueous diluent without the presence of a surfactant or other additive.

TABLE 1
List of stable prednisolone suspensions prepared by microprecipitation process of
FIG. 6 (Example 9)

| | | | 2 Weeks | | 2 Months | | | | | | |
|---|---|---|---|---|---|---|---|---|---|---|---|
| | Initial | | 40° C. | | 5° C. | | 25° C. | | 40° C. | | |
| Formulation | Mean | >99% | Mean | >99% | Mean | >99% | Mean | >99% | Mean | >99% | % Loss* |
| 1.6% prednisolone, 0.6% | 0.79 | 1.65 | 0.84 | 1.79 | 0.83 | 1.86 | 0.82 | 1.78 | 0.82 | 1.93 | <2% |
| phospholipids, 0.5% sodium | | | | | | | | | | | |
| doexycholate, 5 mM TRIS, 2.2% | | | | | | | | | | | |
| glycerol** | | | | | | | | | | | |
| 1.6% prednisolone, 0.6% | 0.77 | 1.52 | 0.79 | 1.67 | 0.805 | 1.763 | 0.796 | 1.693 | 0.81 | 1.633 | <2% |
| Solutol ® , 0.5% sodium | | | | | | | | | | | |
| deoxycholate, 2.2% glycerol | | | | | | | | | | | |
| 1.6% prednisolone, 0.1% | 0.64 | 1.16 | 0.82 | 1.78 | 0.696 | 1.385 | 0.758 | 1.698 | 0.719 | 1.473 | <2% |
| poloxamer 188, 0.5% sodium | | | | | | | | | | | |
| deoxycholate, 2.2% glycerol | | | | | | | | | | | |
| 1.6% prednisolone, 5% | 0.824 | 1.77 | 0.87 | 1.93 | 0.88 | 1.95 | 0.869 | 1.778 | 0.909 | 1.993 | <2% |
| phospholipids, 5 mM TRIS, 2.2% | | | | | | | | | | | |
| glycerol | | | | | | | | | | | |

*Difference in itraconazole concentration between samples stored for 2 months at 5 and 25° C.
**Stable through at least 6 months.
Particle sizes (by laser light scattering), in microns:
5° C.: 0.80 (mean), 1.7 (99%)
25° C.: 0.90 (mean); 2.51 (99%)
40° C.: 0.99 (mean); 2.03 (99%)
Difference in itraconazole concentration between samples stored at 5 and 25° C.: <2%

### Example 10

### Preparation of Prednisolone Suspension by use of Homogenization

32 g of prednisolone was dissolved into 40 mL of NMP. Gentle heating at 40-50° C. was required to effect dissolution. The drug NMP concentrate was subsequently dripped at 2.5 mL/min into 2 liters of a stirred solution that consisted of 0.1.2% lecithin and 2.2% glycerin. No other surface modifiers were added. The surfactant system was buffered at pH=8.0 with 5 mM tris buffer and the temperature was held at 0° C. to 5° C. during the entire precipitation process. The post-precipitated dispersion was next homogenized cold (5-15° C.) for 20 passes at 10,000 psi. Following homogenization, the NMP was removed by centrifuging the suspension, removing the supernatant, and replacing the supernatant with fresh surfactant solution. This post-centrifuged suspension was then rehomogenized cold (5° C.-15° C.) for another 20 passes at 10,000 psi. The particles produced by this process had a mean diameter of 0.927 μm with 99% of the particles being less than 2.36 μm.

### Example 11

### Preparation of Nabumetone Suspension by use of Homogenization

Surfactant (2.2 g of poloxamer 188) was dissolved in 6 mL of N-methyl-2-pyrrolidinone. This solution was stirred at 45° C. for 15 minutes, after which 1.0 g of nabumetone was added. The drug dissolved rapidly. Diluent was prepared which consisted of 5 mM tris buffer with 2.2% glycerol, and adjusted to pH 8. A 100-mL portion of diluent was cooled in an ice bath. The drug concentrate was slowly added (approximately 0.8 mL/min) to the diluent with vigorous stirring. This crude suspension was homogenized at 15,000 psi for 30 minutes and then at 20,000 psi for 30 minutes (temperature=5° C.). The final nanosuspension was found to be 930 nm in effective mean diameter (analyzed by laser diffraction). 99% of the particles were less than approximately 2.6 microns.

### Example 12

### Preparation of Nabumetone Suspension by use of Homogenization and the use of Solutol® HS 15 as the Surfactant. Replacement of Supernatant Liquid with a Phospholipid Medium

Nabumetone (0.987 grams) was dissolved in 8 mL of N-methyl-2-pyrrolidinone. To this solution was added 2.2 grams of Solutol® HS 15. This mixture was stirred until complete dissolution of the surfactant in the drug concentrate. Diluent was prepared, which consisted of 5 mM tris buffer with 2.2% glycerol, and which was adjusted to pH 8. The diluent was cooled in an ice bath, and the drug concentrate was slowly added (approximately 0.5 mL/min) to the diluent with vigorous stirring. This crude suspension was homogenized for 20 minutes at 15,000 psi, and for 30 minutes at 20,000 psi.

The suspension was centrifuged at 15,000 rpm for 15 minutes and the supernatant was removed and discarded. The remaining solid pellet was resuspended in a diluent consisting of 1.2% phospholipids. This medium was equal in volume to the amount of supernatant removed in the previous step. The resulting suspension was then homogenized at approximately 21,000 psi for 30 minutes. The final suspension was analyzed by laser diffraction and was found to contain particles with a mean diameter of 542 nm, and a 99% cumulative particle distribution sized less than 1 micron.

### Example 13

### Preparation of 1% Itraconazole Suspension with Poloxamer with Particles of a Mean Diameter of Approximately 220 nm

Itraconazole concentrate was prepared by dissolving 10.02 grams of itraconazole in 60 mL of N-methyl-2-pyrrolidinone. Heating to 70° C. was required to dissolve the drug. The solution was then cooled to room temperature. A portion of 50 mM tris(hydroxymethyl)aminomethane buffer (tris buffer) was prepared and was pH adjusted to 8.0 with 5M hydrochloric acid. An aqueous surfactant solution was prepared by combining 22 g/L poloxamer 407, 3.0 g/L egg phosphatides, 22 g/L glycerol, and 3.0 g/L sodium cholate dihydrate. 900 mL of the surfactant solution was mixed with 100 mL of the tris buffer to provide 1000 mL of aqueous diluent.

The aqueous diluent was added to the hopper of the homogenizer (APV Gaulin Model 15MR-8TA), which was cooled by using an ice jacket. The solution was rapidly stirred (4700 rpm) and the temperature was monitored. The itraconazole concentrate was slowly added, by use of a syringe pump, at a rate of approximately 2 mL/min. Addition was complete after approximately 30 minute. The resulting suspension was stirred for another 30 minutes while the hopper was still being cooled in an ice jacket, and an aliquot was removed for analysis by light microscopy any dynamic light scatting. The remaining suspension was subsequently homogenized for 15 minutes at 10,000 psi. By the end of the homogenization the temperature had risen to 74° C. The homogenized suspension was collected in a 1-L Type I glass bottle and sealed with a rubber closure. The bottle containing suspension was stored in a refrigerator at 5° C.

A sample of the suspension before homogenization showed the sample to consist of both free particles, clumps of particles, and multilamellar lipid bodies. The free particles could not be clearly visualized due to Brownian motion; however, many of the aggregates appeared to consist of amorphous, non-crystalline material.

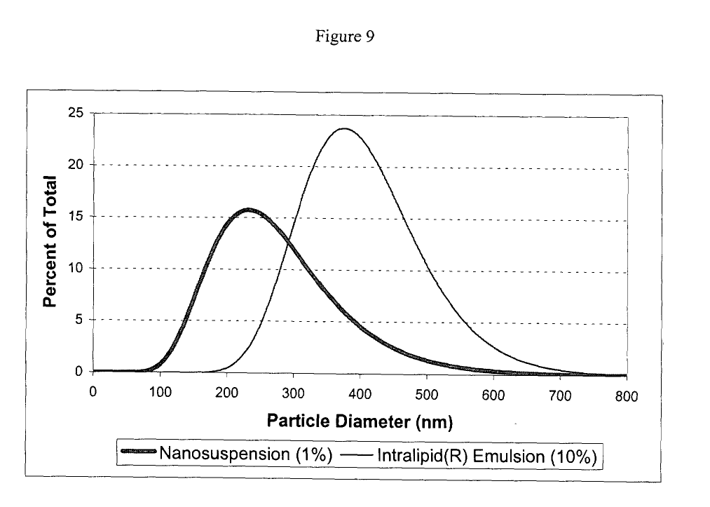

The homogenized sample contained free submicron sized particles having excellent size homogeneity without visible lipid vesicles. Dynamic light scattering showed a monodisperse logarithmic size distribution with a median diameter of approximately 220 nm. The upper 99% cumulative size cutoff was approximately 500 nm. FIG. 9 shows a comparison of the size distribution of the prepared nanosuspension with that of a typical parenteral fat emulsion product (10% Intralipid®, Pharmacia).

### Example 14

### Preparation of 1% Itraconazole Nanosuspension with Hydroxyethylstarch

Preparation of Solution A: Hydroxyethylstarch (1 g, Ajinomoto) was dissolved in 3 mL of N-methyl-2-pyrrolidinone (NMP). This solution was heated in a water bath to 70° C.-80° C. for 1 hour. In another container was added 1 g of itraconazole (Wyckoff). Three mL of NMP were added and the mixture heated to 70-80° C. to effect dissolution (approximately 30 minutes). Phospholipid (Lipoid S-100) was added to this hot solution. Heating was continued at 70-90° C. for 30 minutes until all of the phospholipid was dissolved. The hydroxyethylstarch solution was combined with the itraconazole/ phospholipid solution. This mixture was heated for another 30 minutes at 80-95° C. to dissolve the mixture.

Addition of Solution A to Tris Buffer: Ninety-four (94) mL of 50 mM tris(hydroxymethyl)aminomethane buffer was cooled in an ice bath. As the tris solution was being rapidly stirred, the hot Solution A (see above) was slowly added dropwise (less than 2 cc/minute).

After complete addition, the resulting suspension was sonicated (Cole-Parmer Ultrasonic Processor—20,000 Hz, 80% amplitude setting) while still being cooled in the ice bath. A one-inch solid probe was utilized. Sonication was continued for 5 minutes. The ice bath was removed, the probe was removed and retuned, and the probe was again immersed in the suspension. The suspension was sonicated again for another 5 minutes without the ice bath. The sonicator probe was once again removed and retuned, and after immersion of the probe the sample was sonicated for another 5 minutes. At this point, the temperature of the suspension had risen to 82° C. The suspension was quickly cooled again in an ice bath and when it was found to be below room temperature it was poured into a Type I glass bottle and sealed. Microscopic visualization of the particles indicated individual particle sizes on the order of one micron or less.

After one year of storage at room temperature, the suspension was reevaluated for particle size and found to have a mean diameter of approximately 300 nm.

### Example 15

### Prophetic Example of Preparing 1% Itraconazole Suspension with HES Added to the Aqueous Solution

The present invention contemplates preparing a 1% itraconazole nanosuspension with hydroxyethylstarch by following the steps of Example 14 with the exception the HES would be added to the tris buffer solution instead of to the NMP solution. The aqueous solution may have to be heated to dissolve the HES.

### Example 16

### Seeding During Homogenization to Convert a Less Stable Polymorph to a more Stable Polymorph

Preparation of Starting Material. An itraconazole nanosuspension was prepared by a microprecipitation-homogenization method as follows. Itraconazole (3 g) and Solutol HS 15 (2.25 g) were dissolved in 36 mL of N-methyl-2-pyrrolidinone (NMP) with low heat and stirring to form a drug concentrate solution. The solution was cooled to room temperature and filtered through a 0.2 μm nylon filter under vacuum to remove undissolved drug or particulate matter. The solution was viewed under polarized light to ensure that no crystalline material was present after filtering. The drug concentrate solution was then added at 1.0 mL/minute to approximately 264 mL of an aqueous buffer solution (22 g/L glycerol in 5 mM buffer). The aqueous solution was kept at 2-3° C. and was continuously stirred at approximately 400 rpm during the drug concentrate addition. Approximately 100 mL of the resulting suspension was centrifuged and the solids resuspended in a pre-filtered solution of 20% NMP in water. This suspension was re-centrifuged and the solids were transferred to a vacuum oven for overnight drying at 25° C. The resulting solid sample was labeled SMP 2 PRE.

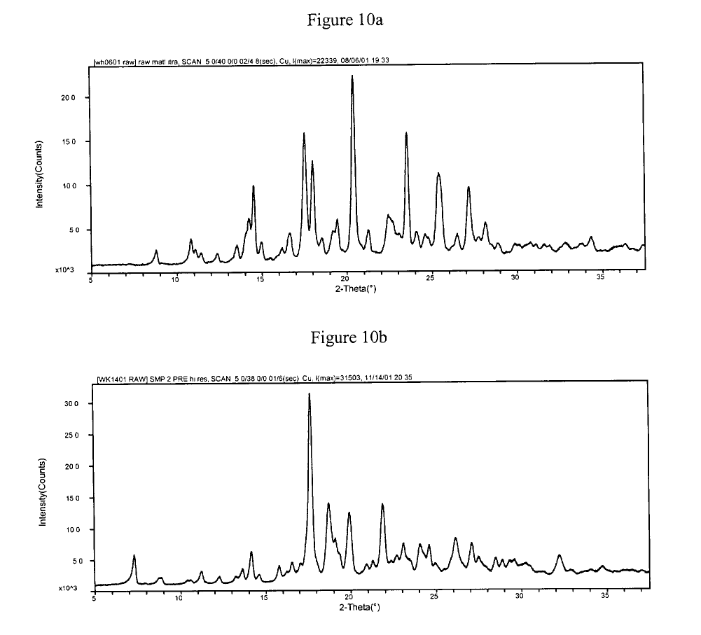

Characterization of Starting Material. The sample SMP 2 PRE and a sample of the raw material itraconazole were analyzed using powder x-ray diffractometry. The measurements were performed using a Rigaku MiniFlex+ instrument with a copper target, Kβ filter, voltage=30 kV and current=40 mA. Data was collected using a step size of 0.02° 2-theta and scan speed of 0.25° 2-theta/minute between 5° and 38° 2-theta. The resulting powder diffraction patterns are shown in FIG. 10. The patterns show that SMP-2-PRE (FIG. 10b) is significantly different from the raw material (FIG. 10a), suggesting the presence of a different polymorph or a pseudopolymorph. The x-ray powder diffraction pattern for SMP-2-PRE exhibits peaks expressed in degrees 2-theta consistent with those listed in Table 3.

TABLE 3
Peaks in degrees 2-theta of the x-ray powder diffraction
pattern of SMP-2-PRE

| 2-Theta | d(A) | I% |
|---|---|---|
| 7.310 | 12.0829 | 16.4 |
| 8.880 | 9.9497 | 4.0 |
| 10.386 | 8.5100 | 1.1 |
| 10.589 | 8.3475 | 2.4 |
| 11.210 | 7.8864 | 6.7 |
| 12.276 | 7.2038 | 3.8 |
| 13.210 | 6.6967 | 2.6 |
| 13.618 | 6.4968 | 6.1 |
| 14.148 | 6.2549 | 15.7 |
| 14.600 | 6.0620 | 3.5 |
| 15.773 | 5.6138 | 6.4 |
| 16.529 | 5.3587 | 6.1 |
| 17.682 | 5.0118 | 100.0 |
| 18.722 | 4.7358 | 37.8 |
| 19.100 | 4.6429 | 17.5 |
| 19.931 | 4.4510 | 32.4 |
| 20.910 | 4.2448 | 3.3 |
| 21.290 | 4.1700 | 4.0 |
| 21.870 | 4.0606 | 34.0 |
| 23.100 | 3.8472 | 13.2 |
| 24.050 | 3.6973 | 13.7 |
| 24.600 | 3.6159 | 14.4 |
| 24.940 | 3.5673 | 2.5 |
| 26.140 | 3.4062 | 17.6 |
| 27.080 | 3.2900 | 14.4 |
| 27.490 | 3.2420 | 6.9 |
| 28.500 | 3.1293 | 6.6 |
| 28.880 | 3.0889 | 4.6 |
| 29.271 | 3.0486 | 3.8 |
| 29.599 | 3.0155 | 4.6 |
| 30.241 | 2.9530 | 3.2 |
| 32.210 | 2.7768 | 10.4 |
| 32.919 | 2.7186 | 1.6 |
| 34.740 | 2.5801 | 3.3 |

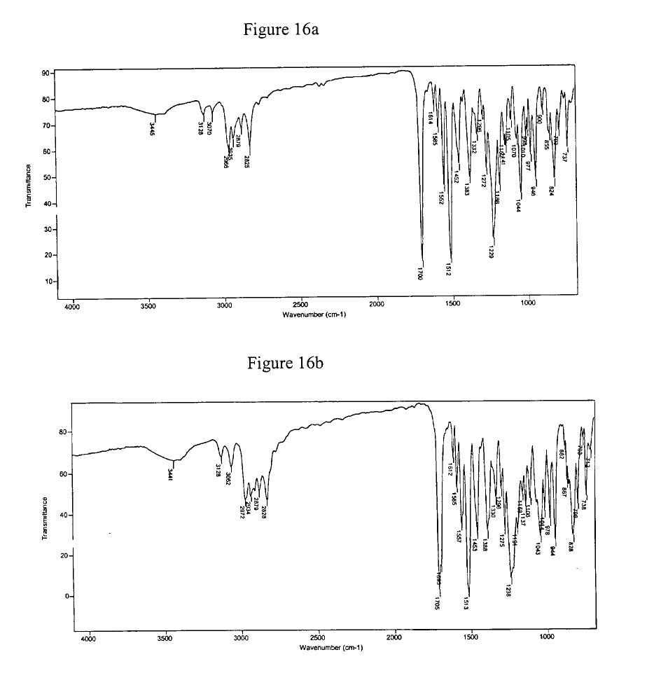

Fourier Transform Infrared (FTIR) spectra of the polymorphic forms of itraconazole are shown in FIGS. 16a and 16b. FIG. 16a is the FTIR spectrum of the raw material of itraconazole, and FIG. 16b is the FTIR spectrum of SMP-2-PRE.

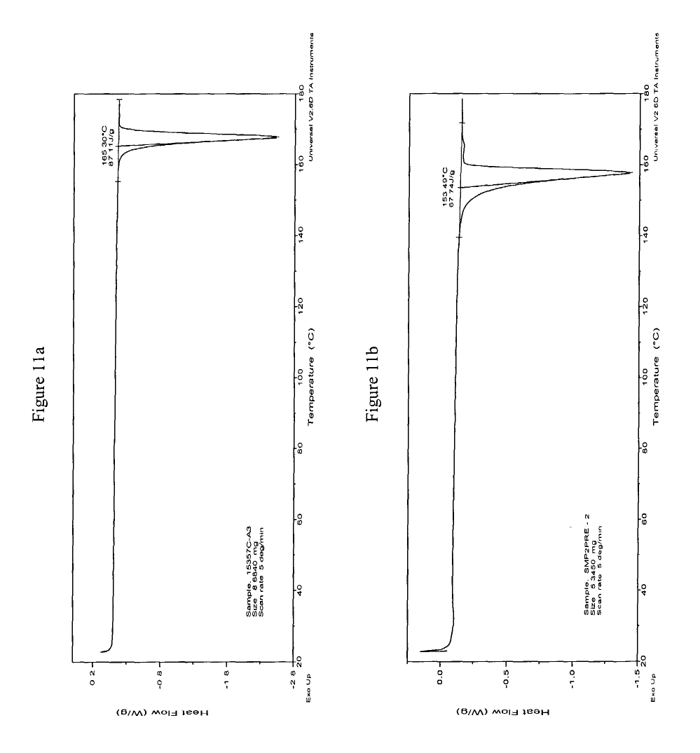

Differential scanning calorimetry (DSC) traces for the samples are shown in FIGS. 11a and 11b. Both samples were heated at 2°/min to 180° C. in hermetically sealed aluminum pans.

The trace for the raw material itraconazole (FIG. 11a) shows a sharp endotherm at approximately 165° C. and an enthalpy of fusion of approximately 87 J/g.

The trace for SMP 2 PRE (FIG. 11b) exhibits an endotherm at approximately 153° C. and an enthalpy of fusion of approximately 68 J/g. This result, in combination with the powder x-ray diffraction patterns and FTIR spectra, suggests that SMP 2 PRE is a new polymorph that is less stable than polymorph present in the raw material.

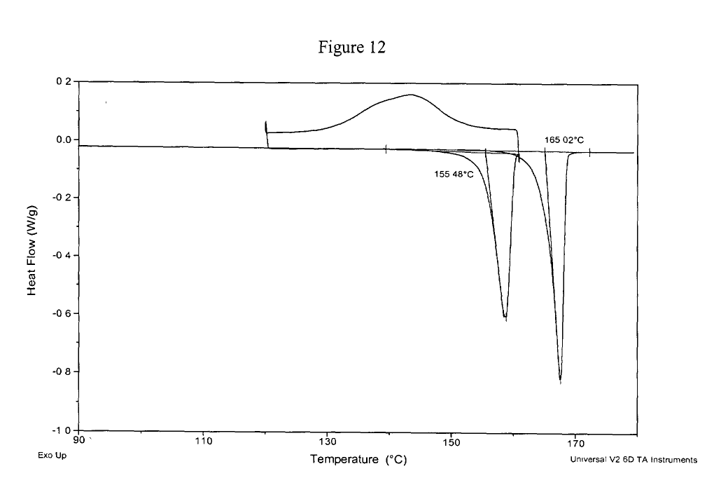

Further evidence for this conclusion is provided by the DSC trace in FIG. 12, which shows that upon heating SMP 2 PRE through the first transition, then cooling and reheating, the less stable polymorph melts and recrystallizes to form the more stable polymorph.

Conversion of Starting Material to a More Stable Polymorph. A suspension was prepared by combining 0.2 g of the solid SMP 2 PRE and 0.2 g of raw material itraconazole with distilled water to a final volume of 20 mL (seeded sample). The suspension was stirred until all the solids were wetted. A second suspension was prepared in the same manner but without adding the raw material itraconazole (unseeded sample). Both suspensions were homogenized at approximately 18,000 psi for 30 minutes. Final temperature of the suspensions after homogenization was approximately 30° C. The approximate size range of the particles in both suspensions was 0.5-2.5 microns, as determined by light microscopy. The suspensions were then centrifuged and the solids dried for approximately 16 hours at 30° C.

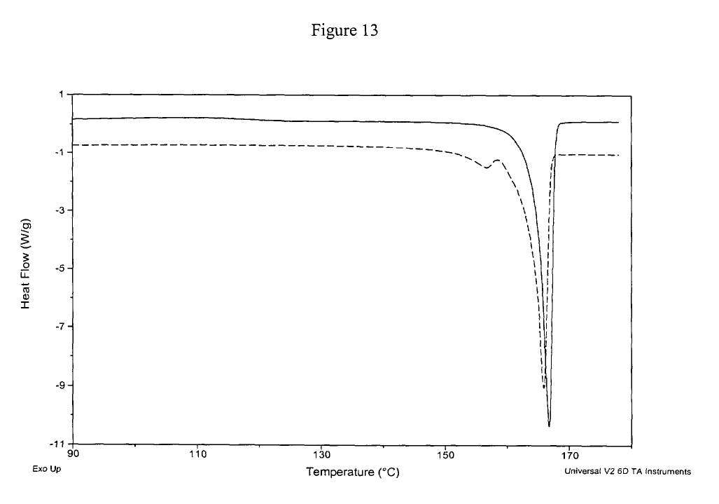

FIG. 13 shows the DSC traces of the seeded and unseeded samples. The heating rate for both samples was 2°/min to 180° C. in hermetically sealed aluminum pans. The trace for the unseeded sample shows two endotherms, indicating that the less stable polymorph is still present after homogenization. The trace for the seeded sample shows that seeding and homogenization causes the conversion of the solids to the stable polymorph. Therefore, seeding appears to influence the kinetics of the transition from the less stable to the more stable polymorphic form.

### Example 17

### Seeding During Precipitation to Preferentially Form a Stable Polymorph

Sample preparation. An itraconazole-NMP drug concentrate was prepared by dissolving 1.67 g of itraconazole in 10 mL of NMP with stirring and gentle heating. The solution was filtered twice using 0.2 ∥m syringe filters. Itraconazole nanosuspensions were then prepared by adding 1.2 mL of the drug concentrate to 20 mL of an aqueous receiving solution at approximately 3° C. and stirring at approx. 500 rpm. A seeded nanosuspension was prepared by using a mixture of approx. 0.02 g of raw material itraconazole in distilled water as the receiving solution. An unseeded nanosuspension was prepared by using distilled water only as the receiving solution. Both suspensions were centrifuged, the supernatants decanted, and the solids dried in a vacuum oven at 30° C. for approximately 16 hours.

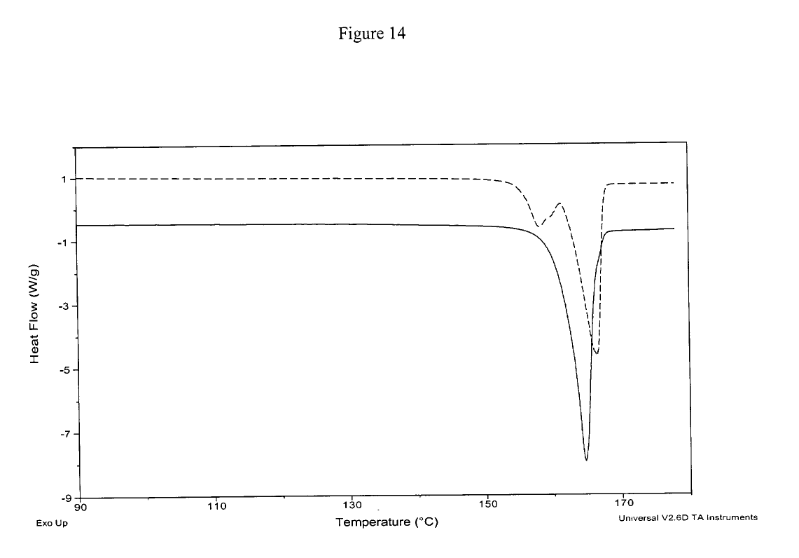

Sample characterization. FIG. 14 shows a comparison of the DSC traces for the solids from the seeded and unseeded suspensions. The samples were heated at 2°/min to 180° C. in hermetically sealed aluminum pans. The dashed line represents the unseeded sample, which shows two endotherms, indicating the presence of a polymorphic mixture.

The solid line represents the seeded sample, which shows only one endotherm near the expected melting temperature of the raw material, indicating that the seed material induced the exclusive formation of the more stable polymorph.

### Example 18

### Polymorph Control by Seeding the Drug Concentrate

Sample preparation. The solubility of itraconazole in NMP at room temperature (approximately 22° C.) was experimentally determined to be 0.16 g/mL. A 0.20 g/mL drug concentrate solution was prepared by dissolving 2.0 g of itraconazole and 0.2 g Poloxamer 188 in 10 mL NMP with heat and stirring. This solution was then allowed to cool to room temperature to yield a supersaturated solution. A microprecipitation experiment was immediately performed in which 1.5 mL of the drug concentrate was added to 30 mL of an aqueous solution containing 0.1% deoxycholate, 2.2% glycerol. The aqueous solution was maintained at —2° C. and a stir rate of 350 rpm during the addition step. The resulting presuspension was homogenized at —13,000 psi for approx. 10 minutes at 50° C. The majority of the particles in suspension were less than 1 micron, as determined by light microscopy. The suspension was then centrifuged, the supernatant decanted, and the solid crystals dried in a vacuum oven at 30° C. for 135 hours.

The supersaturated drug concentrate was subsequently aged by storing at room temperature in order to induce crystallization. After 12 days, the drug concentrate was hazy, indicating that crystal formation had occurred. An itraconazole suspension was prepared from the drug concentrate, in the same manner as in the first experiment, by adding 1.5 mL to 30 mL of an aqueous solution containing 0.1% deoxycholate, 2.2% glycerol. The aqueous solution was maintained at ˜5° C. and a stir rate of 350 rpm during the addition step. The resulting presuspension was homogenized at ˜13,000 psi for approx. 10 minutes at 50° C. The suspension was then centrifuged, the supernatant decanted, and the solid crystals dried in a vacuum oven at 30° C. for 135 hours.

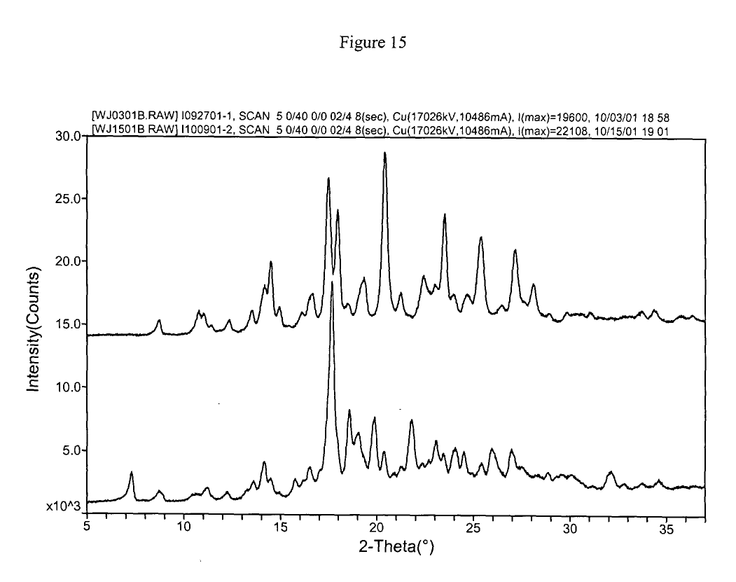

Sample characterization. X-ray powder diffraction analysis was used to determine the morphology of the dried crystals. The resulting patterns are shown in FIG. 15. The crystals from the first experiment (using fresh drug concentrate) were determined to consist of the more stable polymorph. In contrast, the crystals from the second experiment (aged drug concentrate) were predominantly composed of the less stable polymorph, with a small amount of the more stable polymorph also present.

### Example 19

### Prophetic Example of Seeding in an Emulsion Precipitation Method to Form a Stable Polymorph

The present invention contemplates preparing submicron sized particles of a water insoluble compound with a stable polymorph. The method would follow the steps of first dissolving the raw material of the compound in a water immiscible organic solvent to form a solution such that the solution is at or near the saturation point of the compound. This solution is then mixed with an excess of an aqueous solution containing a surface active modifier and a small amount of a stable polymorph of the compound to form a multiphase dispersion of the organic solvent in the aqueous solution. Submicron particles of the stable polymorph of the compound are formed when the multiphase dispersion is frozen and lyophilized to remove the liquid phase of the dispersion. Alternatively, the small amount of the stable polymorph of the compound can be added to the solution of the compound in the organic solvent before the mixing step such that the solubility of the polymorph in the original solution is exceeded, or to the dispersion after the mixing but before the freezing step.

### Example 20

### Seeding During Microprecipitation-Homogenization to Preferentially Form a Stable Polymorph

A 2 liter batch of an itraconazole nanosuspension was prepared as follows. A drug concentrate containing 20 g itraconazole and 2 g Poloxamer 188 in 120 mL N-methyl-2-pyrrolidinone (NMP) was added to an aqueous solution containing 2% mannitol and 0.1% deoxycholic acid sodium salt. This presuspension was then homogenized at 10,000 psi for 15 passes. The suspension was centrifuged to remove the NMP, and the supernatant was replaced with a fresh solution of 0.1% Poloxamer 188, 0.1% deoxycholic acid sodium salt, and 2% mannitol. This suspension was then re-homogenized for an additional 15 passes. A sample of this nanosuspension was tested by x-ray powder diffraction and was found to contain a mixture of polymorphs. Laser diffraction analysis of this suspension gave a mean particle size of 0.43 microns, with 99% of the particles being less than 0.81 microns.

The above process was repeated exactly except with seeding. Seeding was performed by adding approximately 20 mL of an existing nanosuspension to the aqueous solution before the precipitation step. This existing nanosuspension had previously been characterized by x-ray powder diffraction and contained only the stable polymorph. All the subsequent processing steps were then performed as above. A sample of this nanosuspension was tested by x-ray powder diffraction and was found to contain only the stable polymorph. Laser diffraction analysis of this suspension gave a mean particle size of 0.35 microns, with 99% of the particles being less than 0.63 microns.

### Example 21

### Seeding During Microprecipitation-Homogenization to Preferentially Form a Metastable Polymorph

A 10 liter batch of an itraconazole nanosuspension was prepared as follows. An aqueous solution was prepared containing 2.2% glycerol, 0.1% deoxycholic acid sodium salt, and approximately 80 mL of an existing nanosuspension previously characterized by x-ray powder diffraction and found to contain only the metastable polymorph. A drug concentrate containing 100 g itraconazole and 10 g Poloxamer 188 in 1200 mL N-methyl-2-pyrrolidinone (NMP) was added to the aqueous solution to form a presuspension. This presuspension was then homogenized at 10,000 psi for 15 passes. The suspension was centrifuged to remove the NMP, and the supernatant was replaced with a fresh solution of 0.1% Poloxamer 188, 0.1% deoxycholic acid sodium salt, and 2.2% glycerol. This suspension was then re-homogenized for an additional 15 passes. A sample of this nanosuspension was tested by x-ray powder diffraction and was found to contain only the metastable polymorph. Laser diffraction analysis of this suspension gave a mean particle size of 0.20 microns, with 99% of the particles being less than 0.39 microns.

### Example 22

### Conversion of a Metastable Polymorph to a Stable Polymorph through Grinding

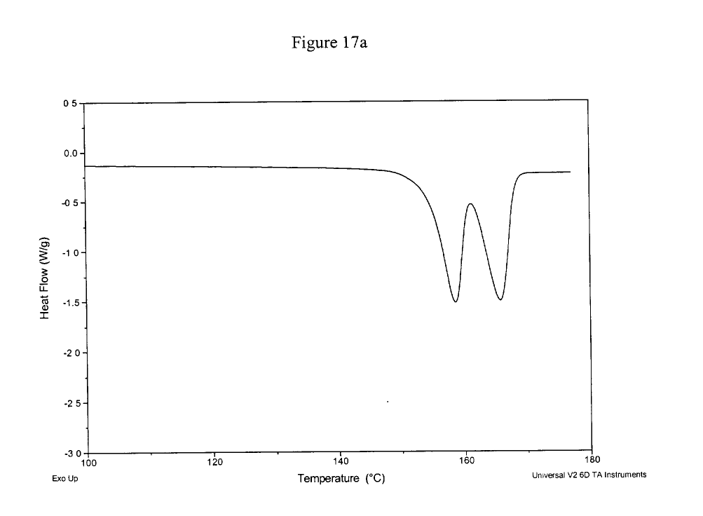

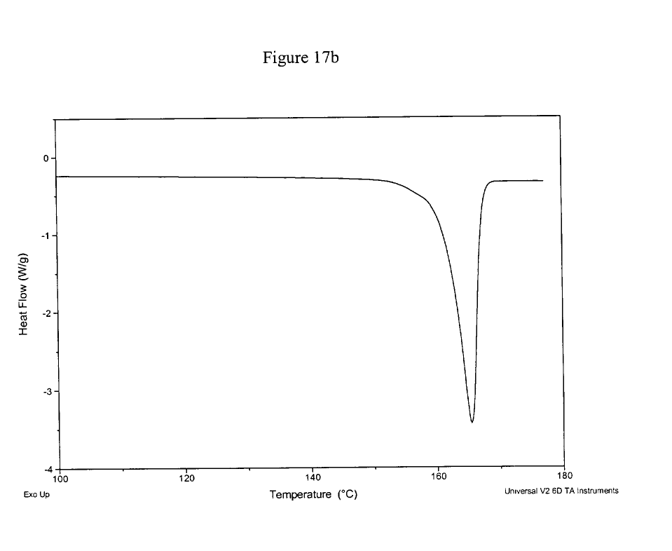

An itraconazole nanosuspension was prepared as follows. A drug concentrate solution was prepared by dissolving 0.625 g Solutol HS and 0.833 g itraconazole in 10 mL of N-methyl-2-pyrrolidinone (NMP) under low heat and stirring. This drug concentrate was filtered using a 0.2 micron syringe filter. A suspension was then prepared by adding 7.2 mL of the filtered drug concentrate to 52.8 mL of an aqueous buffer solution containing 2.2% glycerol and 5 mM Tris. The mixture was maintained at 5° C. and stirred at 400 rpm during the addition. The resulting suspension was centrifuged and the supernatant replaced with a 20% NMP in water solution. The solids were resuspended by shaking and then re-centrifuged. The supernatant was then decanted and the solids dried in a vacuum oven at 25° C. A sample of the resulting powder was analyzed by differential scanning calorimetry (DSC) and found to consist of a mixture of polymorphs. FIG. 17a shows the DSC trace for this sample. A second sample of the powder was ground using a mortar and pestle and then was also analyzed by DSC. The DSC trace for this sample, shown in FIG. 17b, indicates that the majority of the sample is in the more stable polymorphic form. Thus, it is hypothesized that the fraction of the stable form present in the powder before grinding acted as seed material to facilitate the conversion of the metastable material to the stable form.

While specific embodiments have been illustrated and described, numerous modifications come to mind without departing from the spirit of the invention and the scope of protection is only limited by the scope of the accompanying claims.

The invention is claimed as follows:

1. A method of preparing particles with polymorph and size control of a pharmaceutical compound, the method comprising the steps of:
providing a pharmaceutical compound in a first phase;
seeding the compound;
causing a phase change in the pharmaceutical compound to a second phase of a desired polymorphic form; and
wherein the mean particle size of the particles is less than 7 μm.

2. The method of claim 1, wherein the pharmaceutical compound is selected from the group consisting of pharmaceutically active compounds and pharmaceutical excipients.

3. The method of claim 2, wherein the first phase is selected from the group consisting of a supercooled liquid, amorphous, semi-crystalline, and a first polymorphic crystalline form.

4. The method of claim 1, wherein the step of providing a pharmaceutical compound comprises the steps of adding the pharmaceutical compound to a diluent.

5. The method of claim 4, wherein the pharmaceutical compound is soluble in the diluent.

6. The method of claim 4, wherein the pharmaceutical compound is insoluble in the diluent.

7. The method of claim 4, wherein the diluent is a solid a liquid or a compressed gas.

8. The method of claim 4, wherein the step of causing a phase change includes one step selected from the group consisting of precipitating the first compound from the diluent and adding energy to the diluent.

9. The method of claim 5, wherein the step of causing a phase change includes the step of precipitating the pharmaceutical compound from the diluent.

10. The method of claim 9, wherein the step of precipitating the compound is accomplished by a process selected from the group consisting of: microprecipitation, emulsion precipitation, solvent anti-solvent precipitation, phase inversion precipitation, pH shift precipitation, infusion precipitation, temperature shift precipitation, solvent evaporation precipitation, reaction precipitation, and compressed fluid precipitation.

11. The method of claim 10, wherein the diluent is an organic solvent.

12. The method of claim 11, wherein the diluent is water miscible.

13. The method of claim 12, further comprising the step of providing a solution that is aqueous and wherein the step of precipitating the pharmaceutical compound comprises the step of mixing the diluent and pharmaceutical compound with the aqueous solution to form a presuspension.

14. The method of claim 13, wherein the step of seeding comprises the step of seeding at least one of the liquids selected from the group consisting of: the diluent, the aqueous solution and the presuspension.

15. The method of claim 14, wherein the step of mixing comprises the step of adding the diluent and pharmaceutical compound to the aqueous solution.

16. The method of claim 15, further comprising the step of removing the diluent.

17. The method of claim 15, further comprising the step of subjecting the presuspension to high shear mixing.

18. The method of claim 10, wherein the diluent is water immiscible and further comprising the step of providing an aqueous solution.

19. The method of claim 18, further comprising the step of mixing the water immiscible diluent and pharmaceutical compound with the aqueous solution to form an emulsion.

20. The method of claim 19, further comprising the step of removing a portion of the water immiscible diluent from the emulsion to precipitate the pharmaceutical compound.

21. The method of claim 20, wherein the step of seeding comprises the step of seeding a liquid selected from the group consisting of the diluent, the aqueous solution and the emulsion.

22. The method of claim 5, wherein the diluent has a first pH wherein the pharmaceutical compound has a first solubility such that the compound is dissolved in the diluent and wherein the step of precipitating comprises the step of changing the pH of the diluent to a second pH wherein the compound has a second solubility lower than the first solubility and the compound precipitates from the diluent.

23. The method of claim 5, wherein the diluent has a first temperature wherein the pharmaceutical compound has a first solubility such that the compound is dissolved in the diluent and wherein the step of precipitating comprises the step of lowering the temperature of the diluent to a second temperature wherein the compound has a second solubility lower than the first solubility and the compound precipitates from the diluent.

24. The method of claim 5, wherein the step of precipitating comprises the step of removing a portion of the diluent.

25. The method of claim 5, wherein the compound is dissolved in the diluent to form a first solution and wherein the step of precipitating the compound comprises the step of combining the first solution with a compressed fluid.

26. The method of claim 25, wherein the compressed fluid is selected from the group consisting of gas, liquid, or supercritical fluid.

27. The method of claim 1, wherein the step of causing a phase change is by mechanically grinding the compound.

28. The method of claim 27, wherein the step of mechanically grinding is a method selected from the group consisting of: ball milling, pearl milling, hammer milling, fluid energy mills and using a mortar and pestle.

29. The method of claim 6, wherein the pharmaceutical compound is insoluble in the diluent and wherein the step of causing a phase change is by adding energy to the diluent.

30. The method of claim 29, wherein the step of adding energy is accomplished by a process selected from the group consisting of mechanical grinding and high shear mixing.

31. The method of claim 30, wherein the step of mechanical grinding is a process selected from the group consisting of ball milling, pearl milling, dry grinding and wet grinding.

32. The method of claim 30, wherein the step of high shear mixing is carried out using a device selected from the group consisting of a homogenizer, piston gap homogenizer, counter current flow homogenizer, microfluidizer, and sonicator.

33. The method of claim 1, wherein the mean particle size of the compound is less than 3 μm.

34. The method of claim 1, wherein the mean particle size of the compound is less than 1 μm.

35. The method of claim 1, wherein the mean particle size of the compound is less than 500 nm.

36. A method for preparing submicron sized particles of a pharmaceutical compound, the method comprising the steps of:
dissolving the pharmaceutical compound in a first solvent to form a first solution;
precipitating the pharmaceutical compound to form a presuspension;
seeding the first solution or the presuspension; and
wherein the compound in the presuspension is in the form of particles having a mean particle size of less than 7μm and the particles are in a desired polymorphic form.

37. The method of claim 36, wherein the step of precipitating the pharmaceutical compound is by mixing the first solution with a second solvent to precipitate the compound to form the presuspension, and wherein the solubility of the compound is greater in the first solvent than the second solvent.

38. The method of claim 36, further comprising the step of causing a phase change in the pharmaceutical compound from a first phase selected from the group consisting of a supercooled liquid, an amorphous particle, a semicrystalline particle, and a crystalline particle having a first polymorphic form to a second phase of the desired polymorphic form.

39. The method of claim 38, further comprising the step of adding energy to the presuspension.

40. The method of claim 39, wherein the adding-energy step comprises the step of subjecting the presuspension to high shear conditions selected from the group consisting of cavitation, shearing or impact forces utilizing a microfluidizer, piston gap homogenizer or counter current flow homogenizer high energy agitation.

41. The method of claim 39, wherein the adding-energy step comprises the step of adding heat to the presuspension.

42. The method of claim 39, wherein the adding-energy step comprises the step of exposing the presuspension to electromagnetic energy.

43. The method of claim 42, wherein the step of exposing the presuspension to electomagnetic energy comprises the step of exposing the presuspension to a laser beam.

44. The method of claim 38, wherein the step of seeding comprises the step of using a seed compound.

45. The method of claim 44, wherein the seed compound is of the desired polymorphic form of the pharmaceutical compound.

46. The method of claim 44, wherein the seed compound is a compound other than the desired polymorphic form of the pharmaceutical compound.

47. The method of claim 46, wherein the seed compound is selected from the group consisting of: an inert impurity; and an organic compound with a structure similar to that of the desired polymorphic form.

48. The method of claim 44, wherein the seed compound is added to the first solution.

49. The method of claim 44, wherein the seed compound is added to the second solvent.

50. The method of claim 44, wherein the seed compound is added to the presuspension.

51. The method of claim 44, wherein the step of forming a desired polymorph comprises the step of forming a seed compound in the first solution.

52. The method of claim 51, wherein the step of forming the seed compound in the first solution comprises the step of adding the pharmaceutical compound in sufficient quantity to exceed the solubility of the pharmaceutical compound in the first solvent to create a supersaturated solution.

53. The method of claim 51, wherein the step of forming the seed compound in the first solution further comprises the step of treating the first solution.

54. The method of claim 53, wherein the step of treating the supersaturated solution comprises the step of a process selected from the group of aging the supersaturated solution, temperature shifting the solution and pH shifting of the solution.

55. The method of claim 36, wherein the seeding step comprises the step of using electromagnetic energy to form a seed compound.

56. The method of claim 55, wherein the electromagnetic energy is dynamic electromagnetic energy.

57. The method of claim 55, wherein the electromagnetic energy is a laser beam.

58. The method of claim 55, wherein the electromagnetic energy is radiation.

59. The method of claim 36, wherein the step of seeding comprises the step of using a particle beam to form a seed compound.

60. The method of claim 36, wherein the step of seeding comprises the step of using an electron beam to form a seed compound.

61. The method of claim 36, wherein the step of seeding comprises the step of using ultrasound to form a seed compound.

62. The method of claim 36, wherein the step of seeding comprises using a static electric field to form a seed compound.

63. The method of claim 36, wherein the step of seeding comprises using a static magnetic field to form a seed compound.

64. The method of claim 36, wherein the particle has a mean particle size of less than about 2 μm.

65. The method of claim 36, wherein the particle has a mean particle size of less than about 500 nm.

66. The method of claim 36, wherein the particle has a mean particle size of less than 200 nm.

67. The method of claim 37, further including a surface active compound in the presuspension.

68. The method of claim 36, further comprising the step of providing a second solvent and combining the first solvent and the second solvent.

69. The method of claim 68, wherein the second solvent is selected from the group consisting of solvents miscible with the first solvent, and solvents immiscible with the first solvent.

70. The method of claim 69, wherein the first solvent is a water immiscible organic solvent, and the second solvent is an aqueous solution, wherein a multiphase system having an organic phase and an aqueous phase is formed by mixing the organic solvent and the aqueous solution.

71. The method of claim 70, further comprising the step of sonicating the multiphase system to evaporate a portion of the organic phase to cause precipitation of the compound in the aqueous phase to form the presuspension.

72. The method of claim 70, wherein the step of seeding comprises the step of seeding the first solution, the second solvent or the presuspension.

73. The method of claim 70, wherein the step of mixing the water immiscible organic solvent and the aqueous solution to form the multiphase system comprises the use of a piston gap homogenizer, a colloidal mill, high speed stirring, extrusion, manual agitation or shaking, microfluidization, or other high shear conditions.

74. The method of claim 71, wherein the aqueous phase after sonication is essentially free of the organic solvent.

75. The method of claim 70, further comprises the step of evaporating the organic solvent by a method selected from the group consisting of lyophilization, rotary evaporation, and spray drying.

76. The method of claim 36, wherein the step of precipitating is by changing the pH of the first solvent.

77. The method of claim 36, wherein the step of dissolving the compound in the first solvent comprises the steps of bringing the first solvent to a first temperature to form the first solution followed by cooling the first solution to a second temperature to precipitate the compound, wherein the compound is soluble in the first solvent at the first temperature.

78. The method of claim 77, wherein the second temperature is below the melting point of the compound.

79. The method of claim 79, wherein the second temperature is above the melting point of the compound.

80. The method of claim 37, wherein the method of mixing the first solvent with the second solvent is by infusing the second solvent into the first solution to precipitate the compound.

81. The method of claim 80, wherein the first solvent is an organic solvent, and the second solvent is an aqueous solution.

82. The method of claim 37, wherein the second solvent is a compressed fluid.

83. The method of claim 82, wherein the compressed fluid is a supercritical fluid.

84. The method of claim 36, wherein the step of precipitating comprises the steps of causing a reaction of the compound to form a modified compound wherein the modified compound has less solubility in the first solvent than the compound.

85. The method of claim 84, wherein the step of causing a reaction of the first compound comprises the step of adding an agent to chemically react with the first compound or adding energy to cause the first compound to become modified.

86. The method of claim 36, wherein the step of precipitating comprises the step of evaporating a portion of the volume of the first solvent.

87. A method for preparing submicron sized particles of a pharmaceutical compound, the method comprising the steps of:
dissolving the pharmaceutical compound in a first solvent to form a first solution;
mixing the first solution with a second solvent to precipitate the pharmaceutical compound as particles to form a presuspension, wherein the solubility of the pharmaceutical compound is greater in the first solvent than in the second solvent;
providing a seed compound to the first solution or the second solvent or the presuspension;
adding energy to the presuspension; and
wherein the particles have an average particle size of less than 500 nm.

88. The method of claim 87, further comprising the step of forming a desired polymorph of the pharmaceutical compound.

89. The method of claim 88, wherein the seed compound is the desired polymorph of the pharmaceutical compound.

90. The method of claim 88, wherein the seed compound is a compound other than the desired polymorph of the pharmaceutical compound.

91. The method of claim 90, wherein the seed compound is selected from the group consisting of: an inert impurity; and an organic compound with a structure similar to that of the desired polymorph.

92. The method of claim 87, wherein the seed compound is added to the first solution.

93. The method of claim 87, wherein the seed compound is added to the second solvent.

94. The method of claim 87, wherein the seed compound is added to the presuspension.

95. The method of claim 87, wherein the step of forming the desired polymorph comprises the step of forming a seed compound in the first solution.

96. The method of claim 95, wherein the step of forming the seed compound in the first solution comprises the step of adding the pharmaceutical compound in sufficient quantity to exceed the solubility of the pharmaceutical compound in the first solvent to create a supersaturated solution.

97. The method of claim 95, wherein the step of forming the seed compound in the first solution further comprises the step of treating the first solution.

98. The method of claim 97, wherein the step of treating the supersaturated solution comprises the step selected from the group consisting of aging the supersaturated solution, temperature shifting the solution or pH shifting the solution.

99. The method of claim 87 wherein the energy addition step comprises the step of using a device selected from the group consisting of a homogenizer, piston gap homogenizer, counter current flow homogenizer, microfluidizer, milling device and sonicator.

100. A method for preparing submicron sized particles of a pharmaceutical compound, the method comprising the steps of:
adding a sufficient quantity of the pharmaceutical compound to a first solvent to create a supersaturated solution;
aging the supersaturated solution to form detectable crystals to create a seeding mixture; and
mixing the seeding mixture with a second solvent to precipitate the pharmaceutical compound to form a presuspension, wherein the pharmaceutical compound has a greater solubility in the first solvent than in the second solvent.

101. The method of claim 100, further comprising the step of converting the compound in the presuspension to a desired polymorphic form from a first phase selected from the group consisting of a supercooled liquid, an amorphous particle, a semicrystalline particle, and a crystalline particle.

102. The method of claim 101, wherein the step of converting the compound comprises the step of adding energy to the presuspension.

103. The method of claim 102, wherein the adding-energy step comprises the step of adding heat to the presuspension.

104. The method of claim 102, wherein the adding-energy step comprises the step of exposing the presuspension to electromagnetic energy.

105. The method of claim 104, wherein the step of exposing the presuspension to electromagnetic energy comprises the step of exposing the presuspension to a laser beam.

106. The method of claim 102 wherein the step of adding energy comprises the step of using a device selected from the group consisting of a homogenizer, piston gap homogenizer, counter current flow homogenizer, microfluidizer, and sonicator.

107. A method for preparing a submicron sized suspension of a pharmaceutical compound having a desired polymorphic form, the method comprising the steps of:
providing a suitable carrier for the pharmaceutical compound;
dispersing the pharmaceutical compound in the carrier to define a presuspension;
applying energy to the presuspension; and
seeding the presuspension to provide particles of the pharmaceutical compound having an average effective particle size of less than 500 nm and having the desired polymorphic form.

108. The method of claim 107, wherein the applying energy step can be conducted using techniques selected from the group consisting of: mechanical grinding, microfluidization, homogenization and ultrasonication.
